(19) United States
(12) Patent Application Publication
(10) Pub. No.: US 2013/0018112 A1
(43) Pub. Date: Jan. 17, 2013

(54) CELLULOSE NANOPARTICLE AEROGELS, HYDROGELS AND ORGANOGELS

(71) Applicants: Wim Albert Wilfried Irene Thielemans, Nottingham (GB); Rebecca Davies, Nottingham (GB)

(72) Inventors: Wim Albert Wilfried Irene Thielemans, Nottingham (GB); Rebecca Davies, Nottingham (GB)

(73) Assignee: University of Nottingham, Nottingham (GB)

(21) Appl. No.: 13/395,710

(22) PCT Filed: Sep. 14, 2010
(86) PCT No.: PCT/GB2010/051542

§ 371 Date: Oct. 1, 2012

(30) Foreign Application Priority Data
Sep. 14, 2009 (GB) 0916031.8

(51) Int. Cl. C08B 15/00; E04B 1/74; C09D 101/00; B01F 17/32; C09K 3/00; C12N 11/12; A61K 8/72; B01J 31/02; H01B 1/24; A61K 47/38; B82Y 30/00

(52) U.S. Cl. 514/781; 536/56; 252/510; 252/503; 252/506; 252/62; 106/163.01; 516/20; 252/1; 435/179; 502/159; 502/439; 977/773; 977/742

(57) ABSTRACT

A cellulose aerogel comprises a plurality of cellulose nanoparticles. The cellulose nanoparticles preferably comprise at least 50% or 80% cellulose nanocrystals by weight of cellulose nanoparticles, and the cellulose nanoparticle aerogel preferably has a density of from 0.001 to 0.2 g/cm³ or from 0.2 to 1.59 g/cm³ The cellulose nanoparticle aerogel typically has an average pore diameter of less than 100 nmm and the cellulose nanoparticles may comprise anionic and/or cationic surface groups.

This invention relates to the field of cellulose nanoparticle hydrogels, organogels and aerogels, their method of manufacture, and their uses.

Aerogels are porous and nanostructured materials which exhibit unusual properties, such as high porosity and surface area, low density, transparency and low heat conductivity. S. Kistler synthesized the first aerogels in the 1930's. He produced aerogels from a variety of materials such as silica, alumina, rubber and cellulose-derivatives. He also introduced the method for producing aerogels which is still actively used today. To begin, a wet gel (a hydrogel) is formed. This hydrogel undergoes several solvent exchange steps to replace the water with an organic solvent to yield an organogel. The organogel is then dried under supercritical conditions to form the aerogel, a process in which the highly porous structure of the organogel is retained. It follows that an aerogel is a highly porous material composed of many pores that may be empty or filled, or partially filled, with gas (usually air).

A wide variety of aerogels have been reported in the literature. Aerogels can be produced from materials such as silica, alumina, tin oxide, chromia and carbon. Currently, aerogels made from silica are the most widely used. More recently, cellulose has gained interest as a source material for the production of aerogels due to its renewability and biodegradability.

Tan and co-workers produced the first cellulose aerogels (C. Tan, B. M. Fung, J. K. Newman, C. Vu, Adv. Mater., 2001, 13, 644-646). These aerogels displayed bulk densities in the range 0.1-0.35 g/cm³ and specific surface areas of 300 m²/g. Other aerogels typically display porosities up to 99%, bulk densities in the range 0.004-0.500 g/cm³, and surface areas between 600 and 1000 m²/g. The differences in these structural properties can be attributed to the various different production methods and starting materials. The unique properties associated with aerogels have lead to their use in a wide range of applications. Such applications include catalysts, catalyst supports, super-thermal and sound insulators, electronics, particle filters, space and particle research and as a storing media for gases in fuel cells. Cellulose aerogels specifically have proven useful in applications where biocompatibility and biodegradability are important, such as for medicinal, cosmetic and pharmaceutical applications (R. Gavillon, T. Budtova, Biomacromolecules, 2008, 9, 269-277).

Cellulose is the most abundant organic compound found on earth and is widely used due to its availability and sustainable production. An estimated 100 billion metric tonnes of cellulose are produced every year. Cellulose displays very interesting properties such as biodegradability, biocompatibility, chemical and thermal stability and a limited carbon footprint. Cellulose is also a renewable resource.

Cotton may be used as a natural source of cellulose. The amount of hemicellulose, lignin and natural waxes/oils varies from species to species with cotton being almost purely cellulose. Cellulose is fibrillar and is a semi crystalline polymer where the crystalline sections have nanosized dimensions. There are two distinct regions observed within cellulose, the crystalline and the amorphous. In the crystalline regions the chains of β-D-glucopyranosyl units are held together in highly ordered arrays by van der Waals and hydrogen bonds. The remaining amorphous regions display randomly arranged cellulose chains and act as structural defects.

The acid hydrolysis of native cellulose under controlled conditions yields cellulose nanoparticles by preferential hydrolysis of the amorphous regions. The hydronium ions penetrate the amorphous regions in the cellulose and hydrolytically cleave the (1→4) glycosidic bonds (FIG. 2). Mass transfer control (diffusion limitations) could be said to dictate that the amorphous regions will be hydrolysed before the crystalline ones. Therefore, under controlled conditions, such as temperature, time and acid strength, preferentially the glycosidic bonds in the amorphous regions are hydrolytically cleaved leaving the crystalline sections mostly intact.

Acids such as sulphuric acid may be used as the catalyst to catalyse the production of cellulose nanoparticles from cotton. The hydrolysis of cotton typically yields high-aspect ratio nanoparticles that are highly crystalline, >70% crystalline by weight of cellulose nanoparticles, usually >80-85% crystalline. The dimensions of the crystals can vary significantly depending on for instance the time of hydrolysis, source of cellulose and location of cellulose bearing plant growth. The dimensions of the nanoparticles can be controlled by varying the source material of cellulose and the hydrolysis conditions and duration.

The surface of the nanoparticle bears many hydroxyl functions. When the cellulose is hydrolysed in sulphuric acid a fraction of these hydroxyl functions are converted to sulfate groups. The hydroxyl groups enable network formation by hydrogen bonding. The deprotonation of the sulphate groups can result in some repulsion of the nanoparticles and stable dispersions can be formed where the nanoparticles do not touch each other.

A hydrogel is a porous three dimensional solid network in which the pores are filled with water. The first step in the production of aerogels can be the preparation of a hydrogel. In previous work, the cellulose has been dissolved before the starting gel is made. Typically, the source of cellulose has been either cellulose acetate or cellulose carbamate. Innerlohinger used both of these starting materials to produce hydrogels (J. Innerlohinger, H. K. Weber, G. Kraft, Macromol., Symp., 2006, 244, 126-135). The cellulose was first dissolved in N-methyl-morpholine-N-oxide (NMMO) monohydrate and then the NMMO was replaced with water to form a hydrogel. An alternative method of dissolving cellulose acetate was reported by both Tan (C. Tan, B. M. Fung, J. K. Newman, C. Vu, Adv. Mater., 2001, 13, 644-646) and Fischer (F. Fischer, A. Rigacci, R. Pirard, S. Berthon-Fabry, Achard, Polymer, 2006, 47, 7636-7645). In both instances the cellulose acetate was dissolved in acetone. Tan then went on to form the wet gel by crosslinking the cellulose ester with Tolylene-2,4-diisocyanate using pyridine as a catalyst using unesterified hydroxyl groups of the cellulose acetate (Tan et al. also used cellulose acetate butyrate in a similar fashion). Fischer produced their wet gel with a polyphenylpolyisocyanate which acted to form urethane bonds also using the unesterified hydroxyl groups. Other solvents such as aqueous sodium hydroxide and aqueous calcium thiocyanate have also been used to dissolve the cellulose and produce hydrogels. More recently, an alternative solvent was used for the dissolution of cellulose. An ionic liquid, AMIMCl, has been used by Tsioptsias to dissolve the cellulose and form a gel (Tsioptsias, C., Stefopoulos, A., Kokkinomalis, I., Papadopoulou, L., and Panayiotou, C., Green Chemistry, 2008, 10, 965-971). The aerogel was then formed by supercritical CO₂ drying.

As detailed above, the production of aerogels from cellulose requires the use of toxic organic solvents and/or ionic liquids. Accordingly, there exists a need for cellulose-based hydrogels, organogels and aerogels that can be manufactured using benign substances and conditions. Furthermore, it is desirable to provide an aerogel with unique and attractive properties such as easy adaptability, high internal surface area, high porosity, and low density, which also has the advantages of biodegradability and availability of source material associated with known cellulose-based aerogels.

According to a first aspect of the present invention, there is provided a cellulose aerogel comprising a plurality of cellulose nanoparticles.

In the context of the present invention the cellulose aerogel of the first aspect of the invention may also be referred to as a cellulose nanoparticle aerogel. These terms are used interchangeably in the following description.

In the context of this invention the term “nanoparticle” means a particle with at least one dimension, preferably at least two dimensions, of 100 nm or less.

The cellulose nanoparticles may comprise at least 10% cellulose nanocrystals by weight of cellulose nanoparticles, preferably at least 40%, for example at least 50%, cellulose nanocrystals, more preferably at least 60% cellulose nanocrystals, even more preferably at least 70% cellulose nanocrystals, even more preferably at least 80% cellulose nanocrystals and even more preferably at least 85% cellulose nanocrystals. In some embodiments, substantially all of the cellulose nanoparticles may comprise cellulose nanocrystals.

The nanocrystals may have a longitudinal structure. Preferably, the nanocrystals are rodshaped nanocrystals. The nanocrystals may have any predominant shape in cross section perpendicular to the longitudinal axis thereof, such as triangular, square, pentagonal, hexagonal, octagonal, circular, oval, etc. The nanocrystals may have a substantially rectilinear cross-section perpendicular to the longitudinal axis thereof. Of course, any predominant shape is subject to variation within the bounds of crystal morphology, and will include typical irregularities and variation of that shape in any particular nanocrystal. The nanocrystals may in some embodiments have a cross section having one or more sides with a length of from around 1 nm to 90 nm or a cross section having a diameter of from around 1 nm to 90 nm e.g. if the crystal is of ellipsoidal or circular cross section. The length of the nanocrystals may vary substantially. In some embodiments there is a distribution of nanoparticle sizes, average nanoparticle dimensions are typically a cross-sectional dimension, or two cross sectional dimensions, of 1 nm to 90 nm, and a length that is typically much longer, perhaps 100 nm, 500 nm, 1000 nm or longer.

In some embodiments the cellulose nanoparticle aerogel may have a density of from 0.001 to 2 g/cm³, for example 0.001 to 1.59 g/cm³, preferably from 0.001 to 1 g/cm³, more preferably from 0.001 to 0.5 g/cm³, even more preferably from 0.001 to 0.2 g/cm³, most preferably from 0.001 to 0.15 g/cm³. Lower density may be preferable since it results in less solid material and therefore lower heat transfer and possibly more surface area.

In some other embodiments the cellulose nanoparticle aerogel may have a density of from 0.01 to 2 g/cm³, for example 0.01 to 1.59 g/cm³, preferably from 0.05 to 2 g/cm³, more preferably from 0.1 or 0.2 to 2 g/cm³, even more preferably from 1 to 2 g/cm³. Higher density may be beneficial as it can afford a different pore size distribution which can lead to a more preferred pore structure.

In some embodiments the cellulose nanoparticle aerogel may have a porosity of at least 50%, preferably at least 60%, preferably at least 70%, preferably at least 80%, more preferably at least 85%, even more preferably at least 90%, most preferably at least 95%.

In some other embodiments the cellulose nanoparticle aerogel may have a porosity of at most 80%, preferably at most 50%, more preferably at most 30%, even more preferably at most 20%, most preferably at most 10%.

The cellulose nanoparticle aerogel may have a BET (Brunauer-Emmett-Teller) internal surface area of at least 0.1 m²/g, preferably at least 1 m²/g, more preferably at least 100 m²/g, more preferably at least 200 m²/g, more preferably at Least 300 m²/g, even more preferably at least 400 m²/g.

In some embodiments the cellulose nanoparticle aerogel may have an average pore diameter of less than 100 nm, preferably less than 20 nm, more preferably less than 10 nm, even more preferably less than 7 nm, most preferably less than 6 nm. The average pore diameter may be 1 nm or more.

In some other embodiments the cellulose nanoparticle aerogel may have an average pore diameter of more than 1 nm, preferably more than 5 nm, more preferably more than 10 nm, even more preferably more than 20 nm, most preferably more than 30 nm.

In some embodiments the cellulose nanoparticle aerogel may have a multimodal pore size distribution with average pore sizes for each distribution between 0.1 nm and 100 nm.

For some applications, such as the storage of gases, small pore size may be preferable. For catalysis, reagents and products have to be able to reach the catalytic sites and for instance metal nanoparticles need to be able to fit inside the pores.

The cellulose nanoparticles may be derived from plant sources, for example cotton, flax, hemp, jute, straw, or wood. The nanoparticles may alternatively be derived from animals which use cellulose in their body, for example Tunicates. The sizes of the cellulose nanocrystals may depend on the source material from which the nanocrystals are derived.

Preferably, the cellulose nanoparticles are obtained by treating a cellulosic polymer with an acid, such as, for example, phosphoric acid or sulphuric acid or hydrochloric acid, or by oxidative cleavage of the polymer with or without bacteria. Preferably, the cellulose nanoparticles are obtained by treating a cellulosic polymer with an inorganic acid.

Preferably, the cellulose nanoparticles comprise, for example contain or are modified to contain, surface groups which may be anionic, cationic or neutral. In one embodiment, the cellulose nanoparticles comprise anionic and/or cationic surface groups. For example, the cellulose nanoparticles may comprise anionic surface groups. Examples of suitable surface groups for cellulose nanoparticles include, but are not limited to hydroxyl, sulphate, carboxylic acid, nitrate, borate, boronate and amine groups, or combinations thereof. Oxidised cellulose with acid and hydroxyl groups may be utilised to provide a negatively charged aerogel.

The magnitude and nature of the charges on the cellulose nanoparticle surface may be controlled. The surface charge may be controllable by protonation and/or deprotonation of the surface groups, preferably by selective protonation and/or deprotonation of the surface groups. The number of surface groups that are protonated and/or deprotonated may also be controllable.

The cellulose nanoparticle aerogel may exhibit adsorption hysteresis.

The cellulose nanoparticle aerogel may contain one or more gases. Alternatively, the cellulose nanoparticle aerogel may be prepared by degassing a cellulose nanoparticle aerogel containing one or more gases, for example by application of a vacuum. The cellulose nanoparticle aerogel may therefore have areas, for examples one or more pores, that are empty or partially empty of a gas.

The unique properties displayed by carbon nanotubes (CNTs) makes them attractive for incorporation into aerogel structures. In particular, the high electrical conductivity and high mechanical strength exhibited by the CNTs can both enhance the strength of the cellulose aerogel matrix and make it electrically conductive.

CNTs are comprised of hollow cylinders of rolled up graphene sheets. The conductivity of the CNTs is determined by the electronic band structure associated with the chirality of the tube. FIG. 3 shows a schematic of a graphene sheet.

The chiral indices n and m determine the conductivity of the CNT. For n=m tubes there is no band gap observed and the tubes are therefore metallic. When n≠m the tubes will have some band gap and will be semiconducting. For the tubes where n−m=3q (where q is an integer) a small band gap is observed and the tubes are semi-metallic (M. J. O'Connell, Carbon Nanotubes: Properties and Applications, CRC Press, 2006).

Previous work conducted on carbon nanotube aerogels indicates that in a gel environment the CNTs will become cross-linked by van der Waals interactions to form electrically percolating networks. In this previous work, the aerogels were reinforced with PVA (M. B. Bryning, D. E. Milkie, M. F. Islam, L. A. Hough, J. M. Kikkawa, A. G. Yodh, Adv. Mater., 2007, 19, 661-664).

The cellulose nanoparticle aerogel may further comprise one or more carbon nanotubes. The incorporation of CNTs into the cellulose nanoparticle aerogels is advantageous because it can make the aerogel conductive. Furthermore, the presence of an additional reinforcer is not necessary in order to manufacture the aerogel, although the addition of such a reinforcer may be beneficial.

The cellulose nanoparticle aerogel may comprise at least 0.1 wt % carbon nanotubes, at least 0.5 wt % carbon nanotubes, at least 1 wt % carbon nanotubes, or at least 2 wt % carbon nanotubes.

Cellulose nanoparticle aerogels doped with carbon nanotubes may exhibit a conductivity of at least 10⁻¹¹ Scm⁻¹, preferably at least 10⁻¹⁰ Scm⁻¹, more preferably at least 10⁻⁹ Scm⁻¹, even more preferably at least 10⁻⁷ Scm⁻¹, even more preferably at least 10⁻⁵ Scm⁻¹. The level of conductivity may depend on the percolation and/or dispersion of the carbon nanotubes in the aerogel.

The carbon nanotubes may be modified on their surface. This modification may include controlled oxidation to introduce, for example, carboxylic acid groups. The surface modification may improve the dispersibility of the CNTs in the solvent during aerogel manufacturing and/or improve interactions between their surface and the cellulose nanoparticles.

Singularly, CNTs have found application in a wide range of areas such as electronics, energy, sensors, composites and biology. In particular, for use in gas and electrochemical storage, as supercapacitors, molecular computing elements, molecular-filtration membranes, artificial muscles, sensors, heterogeneous catalyst supports and nanotube-blended plastics.

Aerogels doped with CNTs also find application in similar areas to bulk CNTs. Cellulose nanoparticle aerogels doped with CNTs can be used in applications where one or more of biocompatibility, renewability, a degree of biodegradability is required combined with strength and electrical conductivity.

Metal nanoparticles display many appealing properties which make their inclusion into a renewable material such as a cellulose aerogel beneficial. The applications of metal nanoparticles can be extended if they can be suitably supported in an organic matrix material, e.g. a cellulose aerogel, hydrogel or organogel.

Metal nanoparticles display different properties from the bulk metal. The size of the nanoparticles influences their intrinsic properties. Interaction between a support and metal nanoparticles can affect surface Plasmon effects and this can be beneficial (e.g. a shift of response of quantum dots towards visible light so they can be used in photovoltaics). Surface Plasmon effects can be affected by the type of surface groups on the aerogel.

Metal nanoparticles are important in many areas such as electronic, magnetic, mechanical, catalytic and composite materials. Specific applications include sensors, ultrafast data communication, catalytic materials, optical data storage, biological nanosensors, electrochemistry, magnetic, biomedical and optoelectronic nanodevices.

The cellulose nanoparticle aerogel may further comprise one or more metal nanoparticles or metal alloy nanoparticles or metal containing nanoparticles, such as but not limited to gold, silver, palladium and platinum nanoparticles and PbS quantum dots.

The cellulose nanoparticle aerogel may comprise at least 0.1 wt % metal nanoparticles or metal alloy nanoparticles or metal containing nanoparticles, at least 0.5 wt % metal nanoparticles or metal alloy nanoparticles or metal containing nanoparticles, at least 1 wt % metal nanoparticles or metal alloy nanoparticles or metal containing nanoparticles, or at least 2 wt % metal nanoparticles or metal alloy nanoparticles or metal containing nanoparticles. The level of metal nanoparticles or metal alloy nanoparticles or metal containing nanoparticles can be controlled by varying the amount of metal nanoparticle or metal alloy nanoparticle precursor or metal containing nanoparticle precursor or by varying the amount of metal nanoparticles or metal alloy nanoparticles or metal containing nanoparticles that are combined with the cellulose nanoparticles.

Metal can also be deposited inside the pores of the aerogel, hydrogel or organogel in such a way that it coats the cellulose nanoparticles and forms a larger structure than individual nanoparticles. This larger structure can be continuous and stretch through the pores of the hydrogel, aerogel or organogel.

Gold nanoparticles (AuNPs) are the most stable noble metal nanoparticles and display peculiar electronic properties. Silver nanoparticles (AgNPs) exhibit high antimicrobial properties and are used in biological applications such as wound dressing materials, body wall repairs, augmentation devices, tissue scaffolds and antimicrobial filters. Supported platinum nanoparticles (PtNPs) have found application as catalysts for fuel cells and hydrogen storage.

At present, microcrystalline cellulose is used to treat some gastrointestinal diseases and is commonly used as a digestive additive in many pharmaceutical drugs. The medicinal applications of cellulose may be extended with the addition of metal nanoparticles, specifically silver with its antimicrobial properties.

The cellulose nanoparticle aerogels and hydrogels may be useful in the treatment of water or air. For example, hydrogels containing silver nanoparticles can be used to disinfect water, and aerogels containing silver nanoparticles can be used to disinfect air. Aerogels can also work as filters for air.

According to another aspect of the present invention, there is provided a cellulose hydrogel comprising:

a plurality of cellulose nanoparticles and
water.

In the cellulose hydrogel the cellulose nanoparticles may form the major structural component.

According to another aspect of the present invention, there is provided a cellulose organogel, for example an alcogel, comprising:

a plurality of cellulose nanoparticles and
one or more organic solvents.

In the cellulose organogel the cellulose nanoparticles may form the major structural component.

The cellulose nanoparticles of the aerogel, hydrogel or organogel may be chemically cross linked, i.e. connected though chemical linkages, for example at least 5%, preferably at least 10%, more preferably at least 20% of the nanoparticles may be cross linked. Crosslinking may occur between two or more nanoparticles, or sets of nanoparticles, with complementary surface groups (i.e. surface groups that can react with each other). Alternatively or in addition the cellulose aerogel, hydrogel or organogel may include a bifunctional molecule that can react with the surface of the cellulose nanoparticles to give rise to cross linking of the cellulose nanoparticles.

According to another aspect of the present invention, there is provided a method of manufacturing a cellulose nanoparticle aerogel comprising:

(a) forming a cellulose hydrogel comprising a plurality of cellulose nanoparticles or forming a cellulose organogel comprising a plurality of cellulose nanoparticles,
(b) drying the cellulose hydrogel or organogel to form a cellulose nanoparticle aerogel.

The cellulose hydrogel may be formed by mixing a plurality of cellulose nanoparticles in water. The cellulose hydrogel may be dried by freeze drying.

The cellulose organogel may be formed by mixing a plurality of cellulose nanoparticles in an organic solvent. Alternatively the cellulose organogel may be formed by exchanging the water in a cellulose hydrogel comprising a plurality of cellulose nanoparticles with one or more organic solvents. The or each organic solvent must be soluble in water to prepare the organogel by exchange with water from the hydrogel (or vice versa). A second solvent exchange may also be employed to completely or partially replace one or more organic solvents with another one or more organic solvents. The second solvent exchange can occur with one or more organic solvents in which water is not soluble, but which is/are miscible with one or more organic solvents in the starting organogel.

This method has substantial advantages over prior art methods of producing cellulose aerogels because the use of toxic organic solvents and ionic liquids can be avoided as cellulose nanoparticle hydrogels and cellulose nanoparticle organogels are prepared directly from cellulose nanoparticles. This avoids the need to prepare cellulose acetate or cellulose carbamate which, as set out above, requires the use of toxic solvents and liquids. Furthermore the starting materials are renewable.

The mixing may for instance comprise sonication, high shear mixing, stirring, shaking and/or tumbling. The cellulose nanoparticles may be mixed in deionised water to form a hydrogel. In water, the cellulose nanoparticles form a stable physically crosslinked network with one or more of hydrogen bonds, electrostatic hydration forces, and van der Waals forces through the hydroxyl groups and potentially other functional groups on their surface. Additional molecules may be added to improve physical cross linking using one or more of hydrogen bonds, electrostatic hydration forces, and van der Waals forces, or to introduce chemical cross linking by forming chemical linkages between cellulose nanoparticles).

The hydrogel or organogel may be prepared in any suitable concentration of cellulose nanoparticles in water, or one or more organic solvents respectively, such as 3 wt %, 5 wt %, 8 wt %, 10 wt %, 15 wt %, 25 wt % or 50 wt %. The cellulose concentration should be higher than the percolation threshold in the dispersion.

The one or more organic solvents may be any suitable organic solvent, for example any solvent that does not dissolve cellulose, such as one or more of ethanol, methanol, propan-1-ol, propan-2-ol, n-butanol, sec-butanol, iso-butanol, and tert-butanol, acetone, cyclohexane, tetrahydrofuran (THF), N-methylpyrrolidone (NMP), and dimethylformamide (DMF).

The step of mixing the cellulose nanoparticles in water or organic solvent may have a duration of at least 1 min, at least 5 min, at least 15 min, at least 30 min or at least 50 min. The duration of the mixing step may be inversely proportional to the concentration of cellulose nanoparticles in water or organic solvent.

The mixing of the cellulose nanoparticles in water or organic solvent may be carried out at a temperature of less than 50° C., preferably less than 40° C., more preferably less than 30° C., even more preferably less than 25° C.

The point at which a hydrogel or organogel is formed may be determined by the observation of no net movement when a container enclosing the hydrogel or organogel is inverted. The container may be a glass vial mould.

In embodiments where an organogel is formed, the one or more organic solvents of the organogel may be miscible with a supercritical fluid. Where the one or more organic solvents are miscible with a supercritical fluid, the drying of the organogel to form a cellulose nanoparticle aerogel may be performed by supercritical drying with a supercritical fluid. In embodiments where a hydrogel is formed, the hydrogel may be dried using a supercritical fluid that is miscible with water to dry the hydrogel and give rise to the aerogel.

The supercritical fluid may be any suitable supercritical fluid such as one or more of supercritical carbon dioxide, supercritical ethanol, supercritical methanol, supercritical methane, supercritical ethane, supercritical helium. The supercritical fluid cannot be supercritical water.

In relation to exchange of water in a hydrogel for one or more organic solvents to give an organogel, water displays a very poor solubility in some supercritical fluids. Therefore, the water in the pores of a hydrogel can be exchanged with one or more organic solvents in which it is soluble (or several solvent exchange steps can take place as mentioned earlier). The one or more solvents in the final organogel may be miscible in a supercritical fluid e.g. supercritical carbon dioxide. Solvent exchange is a diffusion controlled process. The process depends on factors such as pore size, the size of the gel and the temperature. During this exchange process the gel may undergo an aging procedure which stabilizes and tightens the gel's network.

Alternatively to supercritical drying, step (b) may comprise freeze-drying or subcritically-drying the cellulose hydrogel or organogel to form a cellulose nanoparticle aerogel.

The drying procedure is an important stage in the production of an aerogel. The porous structure present in an organogel or hydrogel should be retained in the aerogel. Accordingly the drying procedure could be any procedure which retains the pore structure of the organogel or hydrogel in the resultant aerogel.

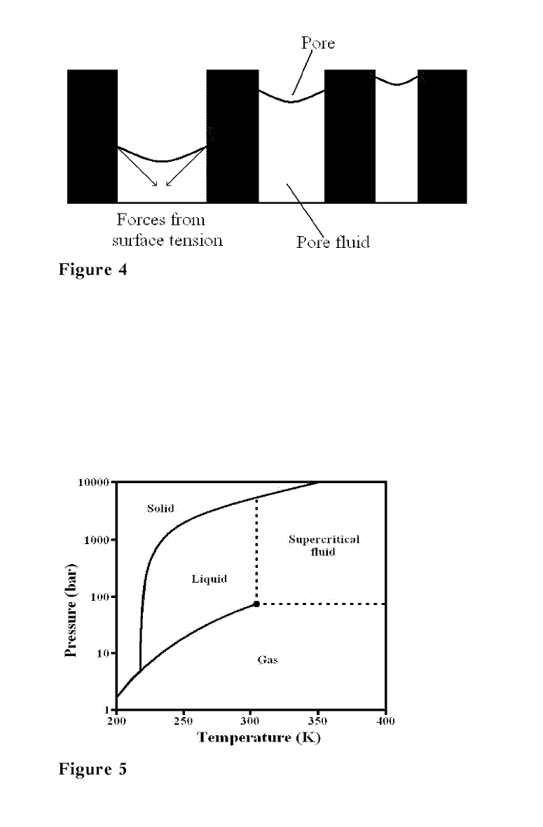

FIG. 4 shows the capillary forces acting on the walls of the pores. As the porous structure is destroyed the internal surface area is also significantly reduced.

To avoid the collapse of the porous structure organogels are commonly dried under supercritical conditions. Under these conditions the surface tension is zero as the liquid-vapour interface is non-existent. Therefore, the absence of capillary forces acting upon the pores allows the material to retain its highly porous structure.

The supercritical fluid usually employed in supercritical drying is carbon dioxide. Carbon dioxide displays favourable properties such as a low cost, non-flammability, low toxicity, low critical point, high availability and high stability.

Carbon dioxide becomes supercritical when it is compressed at a pressure above its critical pressure and at a temperature above its critical temperature as shown in FIG. 5. The supercritical pressure of carbon dioxide is 73.8 bar and the critical temperature is 304.1 K. For example, ethanol within the pores of an organogel forms a mixture with supercritical carbon dioxide (scCO₂) at a temperature of 40° C.

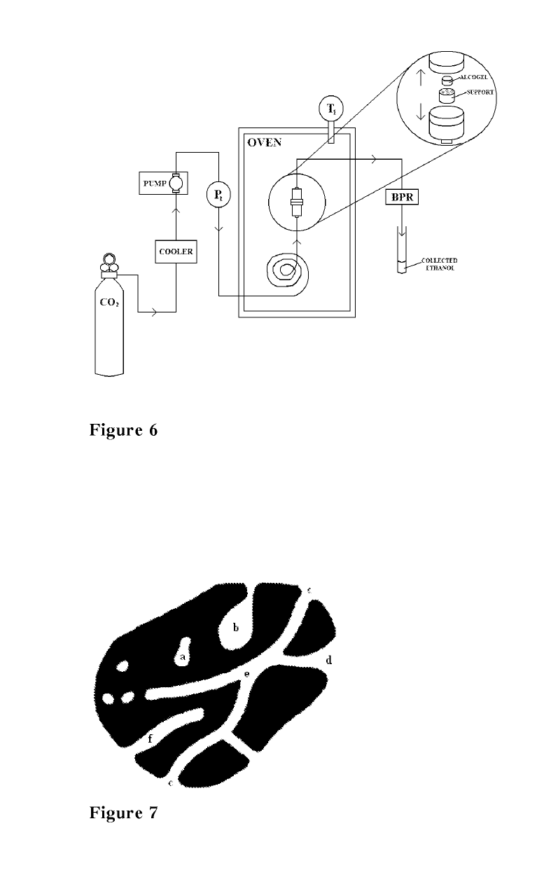

The supercritical drying may be carried out using a supercritical drying rig. A supercritical drying rig is shown in FIG. 6. The supercritical drying rig should comprise a high pressure autoclave. The supercritical drying may be carried out at a temperature and pressure dependent on the supercritical fluid used. For example, when using carbon dioxide the temperature may be 40° C. and the pressure 10 000 kPa (100 bar). The supercritical drying may be carried out for a duration of at least 10 min, at least 30 min, at least 1 hr, at least 2 hr, at least 4 hr or at least 6 hr depending on the organogel to be dried. The required duration may depend on the temperature, the pressure, the nature of the organic solvent in the organogel and the flow rate of the supercritical fluid, the supercritical fluid used, and also pore structure of the hydrogel/organogel.

The method of manufacturing a cellulose nanoparticle aerogel may be carried out in the presence of one or more carbon nanotubes to form a cellulose nanoparticle aerogel comprising one or more carbon nanotubes. The method may comprise, in step (a), mixing a plurality of cellulose nanoparticles and one or more carbon nanotubes in water to form a cellulose hydrogel comprising one or more carbon nanotubes, which can if necessary be subjected to solvent exchange to form an organogel, or mixing a plurality of cellulose nanoparticles and one or more carbon nanotubes in one or more organic solvents to form a cellulose organogel comprising one or more carbon nanotubes.

The mixing may for instance comprise sonication, stirring, high shear mixing, shaking and/or tumbling. The one or more carbon nanotubes may be dispersed in water or one or more organic solvents prior to mixing with the plurality of cellulose nanoparticles. The dispersion of the one or more carbon nanotubes in water or one or more organic solvents may be achieved by sonication, stirring, high shear mixing, shaking and/or tumbling. The dispersion may be aided by surface modification of the carbon nanotubes to make them more dispersible in water or one or more organic solvents.

The method of manufacturing a cellulose nanoparticle aerogel may be carried out in the presence of one or more metal nanoparticles, metal alloy nanoparticles and/or metal containing nanoparticles to form a cellulose nanoparticle aerogel comprising one or more metal nanoparticles, metal alloy nanoparticles and/or metal containing nanoparticles. The method may comprise, in step (a), mixing a plurality of cellulose nanoparticles and one or more metal nanoparticles, metal alloy nanoparticles and/or metal containing nanoparticles in water to form a cellulose nanoparticle hydrogel comprising one or more metal nanoparticles, metal alloy nanoparticles and/or metal containing nanoparticles, which can if necessary be subjected to solvent exchange to form an organogel, or mixing a plurality of cellulose nanoparticles and one or more metal nanoparticles, metal alloy nanoparticles and/or metal containing nanoparticles in one or more organic solvents to form a cellulose nanoparticle organogel comprising one or more metal nanoparticles, metal alloy nanoparticles and/or metal containing nanoparticles.

The mixing may for instance comprise sonication, stirring, high shear mixing, shaking and/or tumbling. Metal nanoparticles may be synthesized before incorporation into a hydrogel or organogel. The highly porous nature of a hydrogel or organogel combined with the strength of the interconnected network and large surface area allows a high loading content of metal nanoparticles.

Alternatively, in another approach the cellulose aerogel acts both as a nanoreactor and a stabilizer. The metal nanoparticles are produced in situ with the cellulose acting as the reducing agent. Without being limited to this hypothesis it is generally thought that metal ions in the form of metal complexes, acids or salts are chemically reduced by both the reducing end groups and hydroxyl groups of cellulose and possibly also during the drying process. Once formed, the metal nanoparticles are prevented from aggregating by the highly ordered structure of cellulose hydrogels/organogels. Additional reducing agents, for example, NaBH₄, H₂, etc., can be added during the reduction and/or drying process to guarantee maximum and/or complete reduction of the metal precursors. The reduction process can also result in a larger metal structure covering the internal surface of the cellulose organogel or hydrogel. The cellulose does not need to participate in the reduction process if additional reducing agent(s) is used.

The method may therefore comprise, after step (a), contacting the hydrogel or organogel with one or a mixture of metal complex(es), acid(s) or salt(s) to form a cellulose hydrogel comprising a plurality of cellulose nanoparticles and one or more metal nanoparticles, metal alloy nanoparticles or metal containing nanoparticles; or adding one or a mixture of metal complex(es), acid(s) or salt(s) during or before step (a) to give rise to a hydrogel or organogel comprising a plurality of cellulose nanoparticles and one or more metal nanoparticles, metal alloy nanoparticles and/or metal containing nanoparticles.

The step of contacting the hydrogel or organogel with the one or mixture of metal complex(es), acid(s) or salt(s) may be carried out for at least 10 min, at least 30 min, at least 1 hr, at least 5 hr, at least 15 hr, or at least 24 hr. A reducing agent such as NaBH₄, or H, may be added during this step. The addition of a reducing agent may not be necessary but can lead to faster and more thorough reduction. In other instances, the addition of a reducing agent is necessary. Furthermore, some of the reduction may occur during supercritical drying due to the use of higher pressure and temperature, and extra reducing agent may be added to the supercritical fluid.

The method may further comprise chemically modifying the surface of the aerogel. This enables the introduction of any suitable functionality utilising known cellulose surface chemistry before and/or after aerogel, organogel or hydrogel formation.

The method may further comprise addition of a bifunctional molecule that reacts with the surface of the cellulose nanoparticles during or after formation of any of the hydrogel, organogel or aerogel. This has the effect of cross linking the nanoparticles within the hydrogel, organogel or aerogel.

According to a further aspect of the present invention, there is provided the use of a cellulose nanoparticle aerogel, hydrogel or organogel according to the invention as, or in a material for use as, a thermal, sound or electrical insulator, a catalyst, a catalyst support, an electronics component, a particle filter, a storage media for substances, a coating, a rheology modifier, a thickening agent, a controlled release agent, an IR suppressant, a surface functionality to bind metals to provide renewable porous catalyst supports, a scaffold for cell growth, in drug delivery, medicinal, water treatment, air disinfection or purification or filtration, gas adsorption or separation, metal sequestration or cosmetic applications, or in space and particle research. There is also provided use of a cellulose nanoparticle aerogel, hydrogel or organogel according to the invention in textiles and outdoor apparel, sports equipment, in the absorption of materials, for example oil, and for use in or as windows.

Other uses of the cellulose nanoparticle aerogel, hydrogel or organogel are also envisaged.

The use of a cellulose nanoparticle aerogel as, or in a material for use as, a storage media for substances may be in a fuel cell.

According to a further aspect of the present invention, there is provided the use of a cellulose nanoparticle aerogel, hydrogel or organogel comprising one or more carbon nanotubes in gas and electrochemical storage media, supercapacitors, molecular computing elements, molecular-filtration membranes, artificial muscles, sensors, heterogeneous catalyst supports or nanotube-blended plastics.

Other uses of the cellulose nanoparticle aerogel, hydrogel or organogel comprising one or more carbon nanotubes are also envisaged.

According to a further aspect of the present invention, there is provided the use of a cellulose nanoparticle hydrogel, organogel or aerogel comprising one or more metal nanoparticles or metal nanoparticle alloys or metal containing nanoparticles or metal structures, metal alloy structures or metal containing structures in electronic, magnetic, mechanical, catalytic or composite materials; sensors; ultrafast data communication; catalytic materials; photovoltaics; optical data storage; biological (nano)sensors; electrochemistry; magnetic, biomedical or optoelectronic (nano)devices; medical, antimicrobial or biological applications such as wound dressing materials, body wall repairs, augmentation devices, tissue scaffolds or antimicrobial filters; catalysts for fuel cells; pharmaceutical drug additives; the storage of substances such as hydrogen; the capture and storage of substances such as carbon dioxide; water treatment; or air purification/disinfection, insulation. The storage of substances or the capture and storage of substances may occur via gas adsorption (with or without the spillover effect).

In particular cellulose nanoparticle organogels, with or without one or more metal nanoparticles or metal nanoparticle alloys or metal containing nanoparticles or metal structures, metal alloy structures or metal containing structures, can be used in drug delivery, pharmaceuticals, cosmetics, art conservation, food, sunscreens, wound care, nutraceuticals, synthesis of nanoscale materials, molecular scaffolds, molecular electronics, fluorescence based applications.

In particular cellulose nanoparticle hydrogels, with or without one or more metal nanoparticles or metal nanoparticle alloys or metal containing nanoparticles or metal structures, metal alloy structures or metal containing structures, can be used in: water disinfection, scaffolds for biological applications including tissue engineering, environmental sensing, drug-delivery systems, biosensors, further biological tissue applications, nappies, contact lenses, medical electrodes, water gel explosives, wound care, breast implants, granules for moisture retention, horticulture, hygiene products, drug screening, pathogen detection, medical devices, other biomedical applications, lubrication (surface coating), sensors, seals, medical devices, biocatalysts.

In particular cellulose nanoparticle aerogels comprising one or more metal nanoparticles or metal nanoparticle alloys or metals containing manoparticles or metal structures, metal alloy structures or metal containing structures can be used in air purification, gas storage/separation, air disinfection, insulation.

Other uses of the cellulose nanoparticle aerogel, hydrogel and organogel comprising one or more metal nanoparticles or metal nanoparticle alloys or metal containing nanoparticles or metal structures, metal alloy structures or metal containing structures are also envisaged.

According to another aspect of the present invention, there is provided a container enclosing a cellulose nanoparticle aerogel, hydrogel or organogel according to the invention.

The container may further enclose one or more substances. A proportion or all of the one or more substances may be releasably stored by the material.

Such a container can be advantageously utilised in fuel cells, batteries, electronics, chemical storage and sensors.

According to another aspect of the present invention, there is provided a renewable porous catalyst support comprising a cellulose nanoparticle aerogel, organogel or hydrogel according to the invention with metallic groups, organic catalysts or enzymes immobilised on or into the aerogel, organogel or hydrogel. Suitable metallic groups could be metals, metallic complexes or organometallic compounds. Organic catalysts could be sulphur ylids or organic acids. Enzymes could be for example lipases.

These renewable porous catalyst supports containing metal centres can act as catalysts in a similar fashion as the metal centres in zeolites. Pore size restrictions may be similar to those of zeolites.

Reusable porous catalyst supports are advantageous in view of their environmental and financial benefits.

According to another aspect of the present invention, there is provided a particle filter comprising a cellulose nanoparticle aerogel according to the invention.

Such a particle filter can advantageously be tailored with a particular pore size to suit a given use such as air treatment by incorporating antiviral or antibacterial agents such as quaternary ammonium salts or silver nanoparticles into the aerogel.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

The cellulose nanoparticle aerogels can take any suitable form, for example they may be monolithic, solid blocks, or particles.

The aerogels, hydrogels and organogels of the present invention may comprise one or more other suitable substance in addition to cellulose nanoparticles and, in the case of hydrogels and organogels one of water and an organic solvent respectively. The or each additional substance may be a polymer, for example water soluble polymers such as polyvinyl alcohol or polyethylene oxide. The or each additional substance may introduce functionality into the aerogel, hydrogel or organogel. The additional substances may be added during the preparation process at any suitable point.

An embodiment of the present invention will now be described herein, by way of example only, with reference to the following figures:

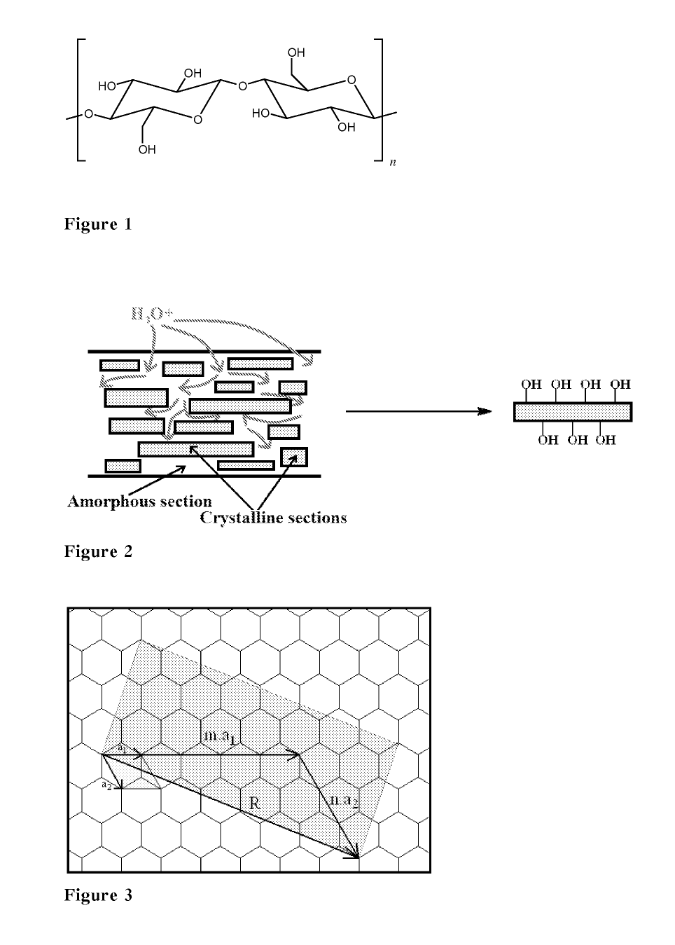

FIG. 1—shows the structure of the cellulose biopolymer;

FIG. 2—shows a schematic of the acid hydrolysis of cellulose;

FIG. 3—shows a schematic of a graphene sheet showing the lattice vectors a₁ and a₂, the roll up vector R and the chiral indices n and m;

FIG. 4—shows a representation of the contracting surface forces in the pores caused by surface tension of the leaving solvent;

FIG. 5—shows a carbon dioxide phase diagram;

FIG. 6—shows a schematic representation of the supercritical drying rig where Pᵢ is the pressure monitor, Tᵢ is the thermocouple and BPR is the Back Pressure Regulator;

FIG. 7—shows a schematic diagram representing the different pore types. (a) represents a closed pore which is totally isolated from its neighbouring pores. (b), (c), (d), (e) and (f) represent open pores which are all connected to the external surface of the material;

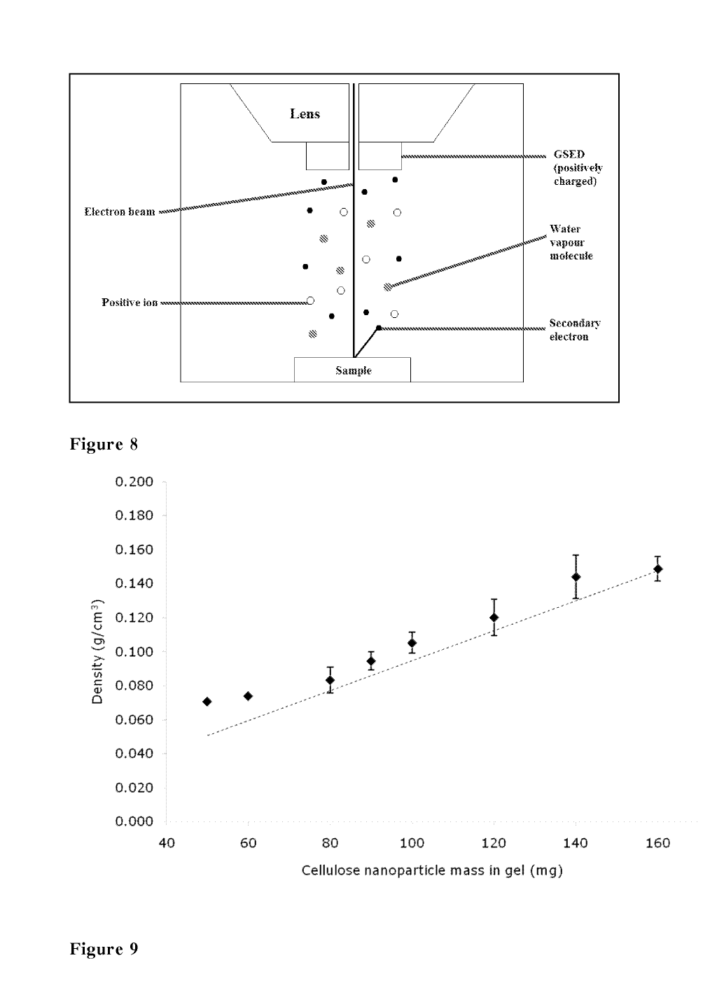

FIG. 8—shows a schematic diagram of a gaseous secondary electron detector (GSED);

FIG. 9—shows the effect of an increasing cellulose nanoparticle concentration on the density of aerogels of the present invention;

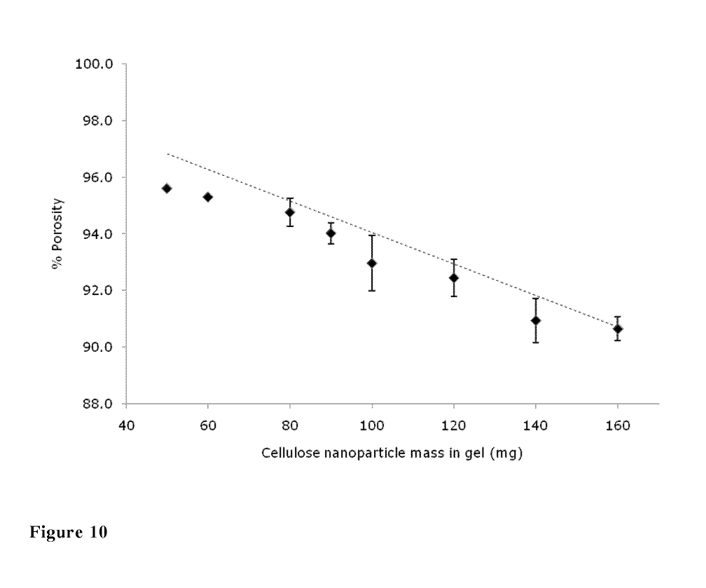

FIG. 10—shows the effect of an increasing cellulose nanoparticle concentration on the percentage porosity of aerogels of the present invention;

FIG. 11—shows the X-ray diffractograms for the cellulose nanoparticles and aerogel samples of the present invention;

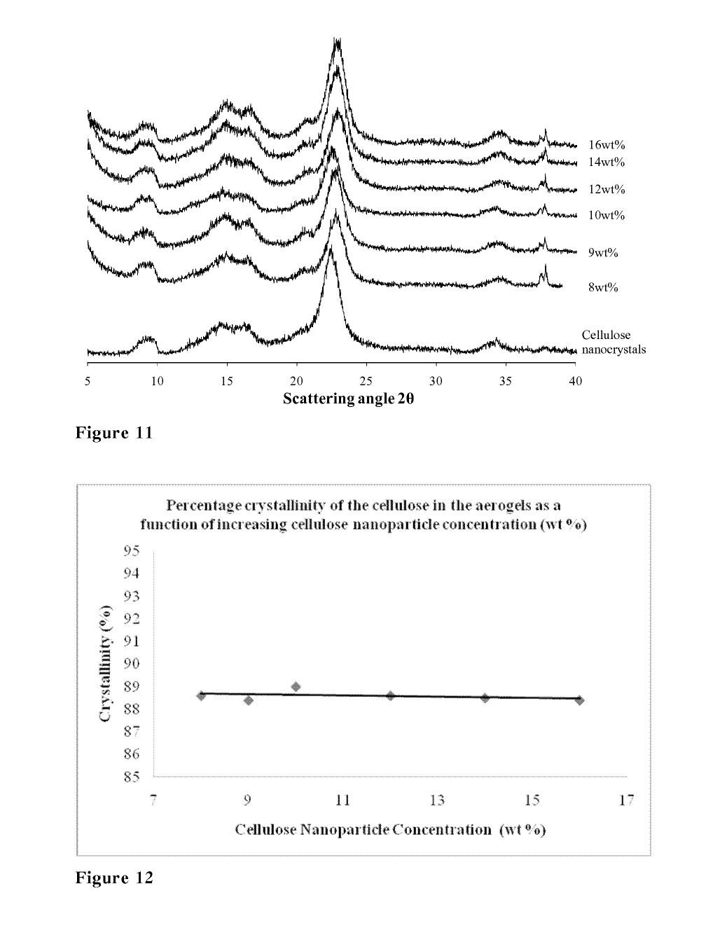

FIG. 12—shows RCI values expressed as a function of increasing cellulose nanoparticle concentration;

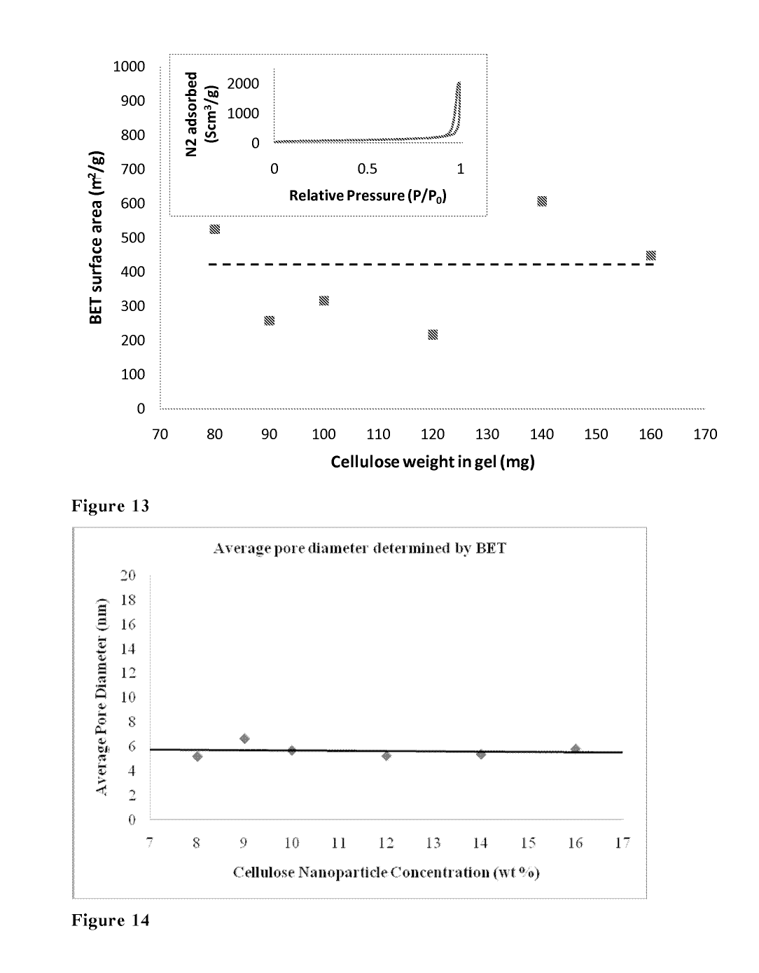

FIG. 13—shows BET surface area values for aerogels of the present invention displaying increasing cellulose nanoparticle concentrations;

FIG. 14—shows the average pore size calculated by BET for aerogels of the present invention;

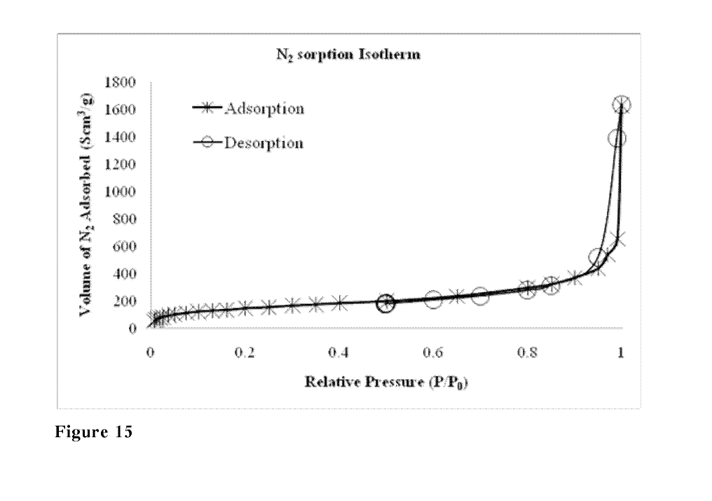

FIG. 15—shows a typical nitrogen sorption isotherm for a cellulose nanoparticle aerogel of the present invention;

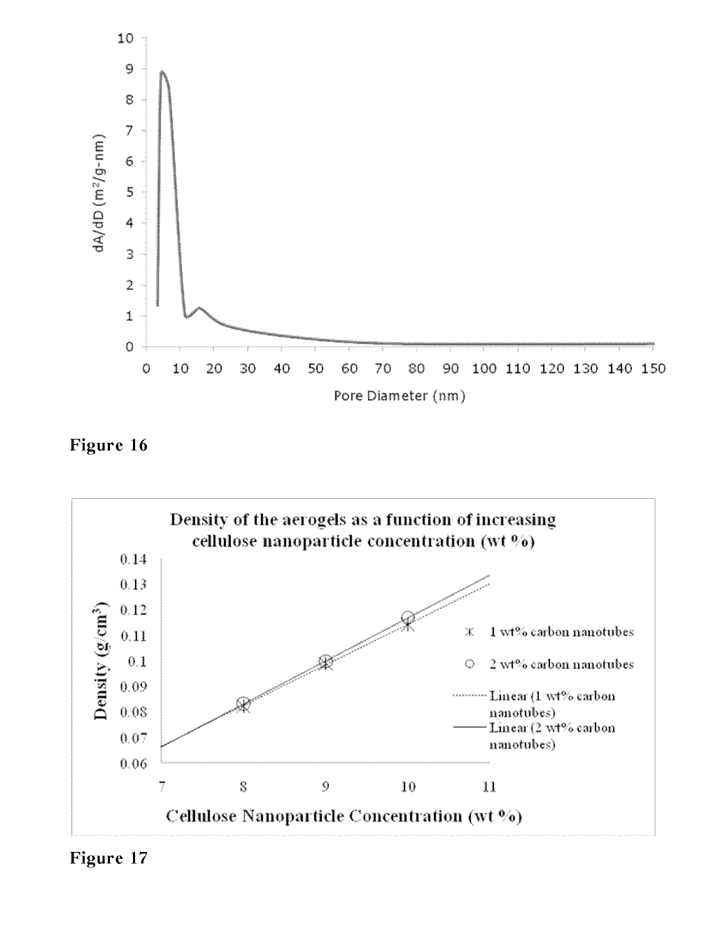

FIG. 16—shows the pore size distribution obtained from the adsorption curve of the isotherm for an 8 wt % aerogel of the present invention;

FIG. 17—shows the effect of an increasing cellulose nanoparticle and carbon nanotube concentration on the density of the aerogels of the present invention;

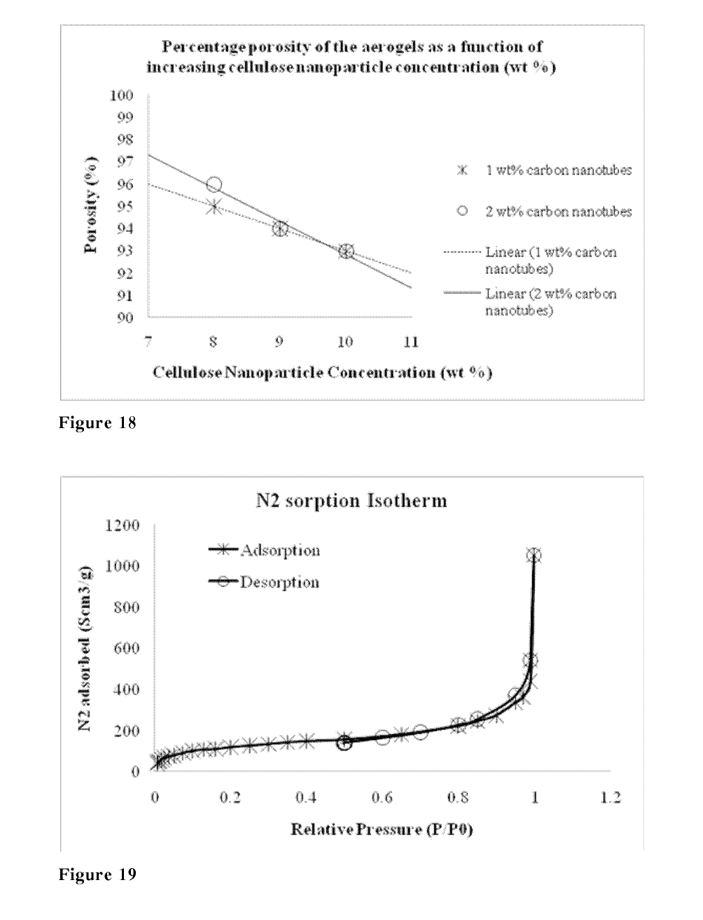

FIG. 18—shows the effect of an increasing cellulose nanoparticle and carbon nanotube concentration on the percentage porosity of the aerogels of the present invention;

FIG. 19—shows a typical nitrogen sorption isotherm for a carbon nanotube doped cellulose nanoparticle aerogel of the present invention;

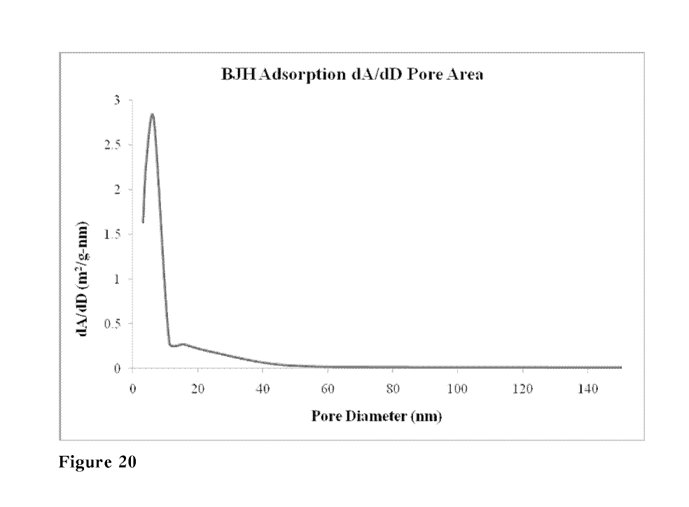

FIG. 20—shows the pore size distribution obtained from the adsorption curve of the isotherm for a 8 wt % aerogel of the present invention doped with 1 wt % carbon nanotubes;

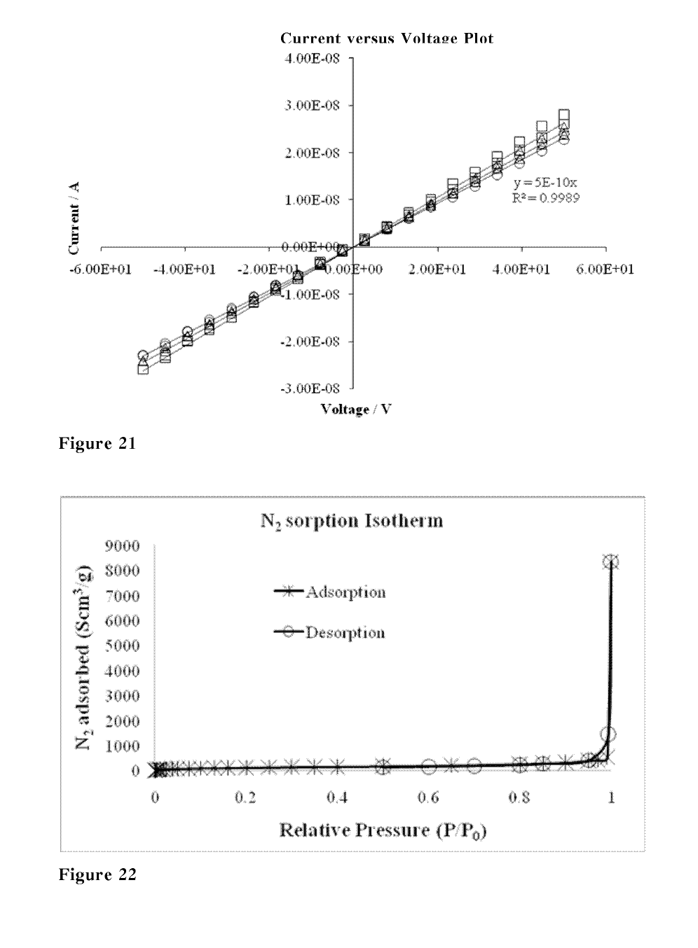

FIG. 21—shows the current versus voltage plot for an aerogel of the present invention with a 9 wt % cellulose nanoparticle concentration and a 1 wt % carbon nanotube concentration. Three sets of data were plotted (squares, triangles and circles) for the conducting aerogel and the linear fit data values were used to calculate conductivity;

FIG. 22—shows a nitrogen sorption isotherm for a AuNP doped 8 wt % cellulose nanoparticle aerogel of the present invention;

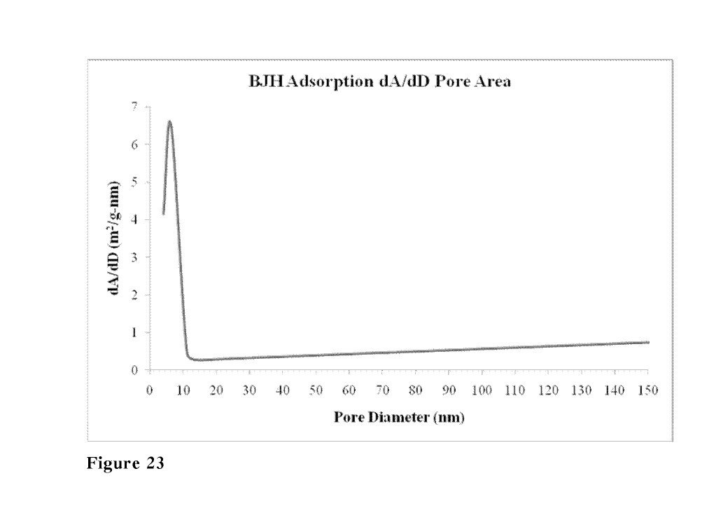

FIG. 23—shows the pore size distribution obtained from the adsorption curve of the isotherm for an AuNP doped 8 wt % cellulose nanoparticle aerogel of the present invention;

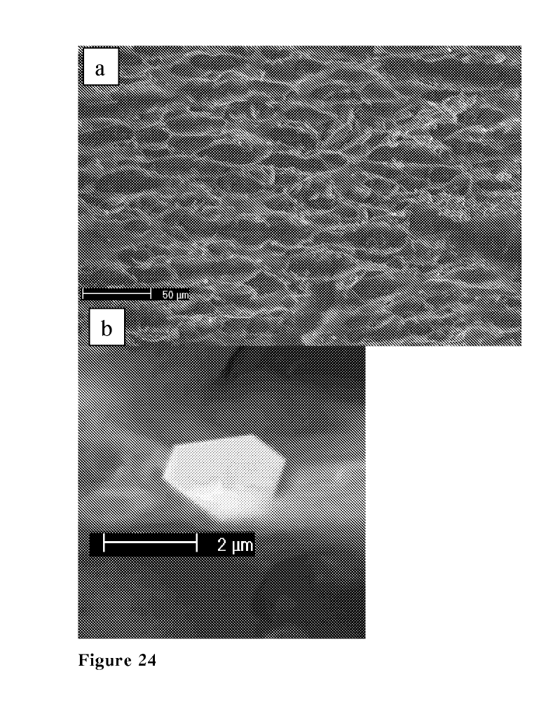

FIG. 24—shows ESEM images of an AuNP doped 8 wt % cellulose nanoparticle aerogel of the present invention;

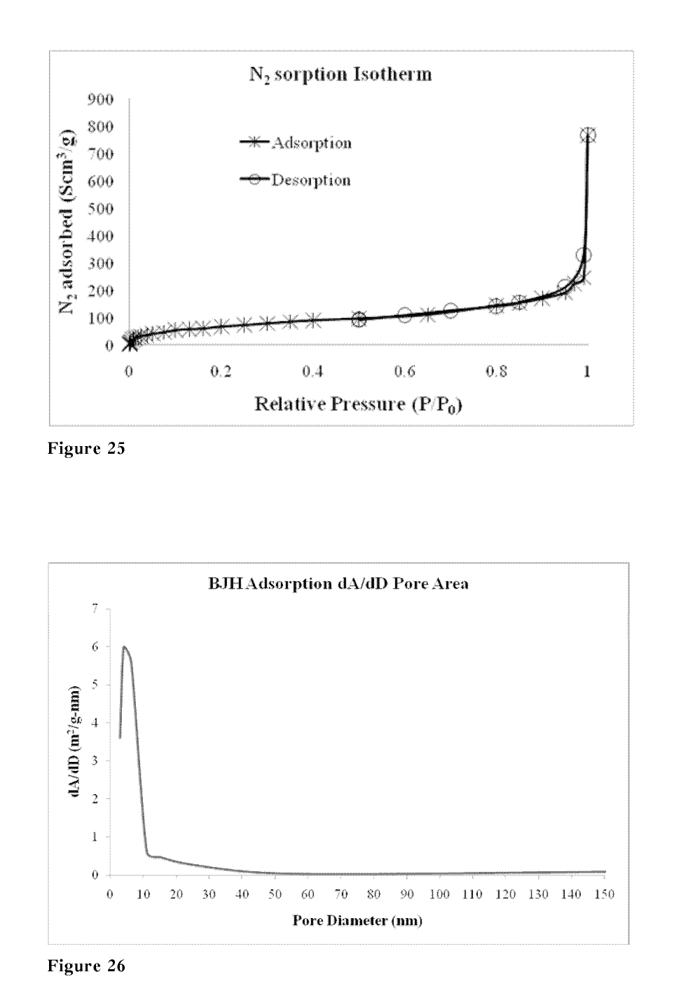

FIG. 25—shows a nitrogen sorption isotherm for a AgNP doped 8 wt % cellulose nanoparticle aerogel of the present invention produced using procedure 2;

FIG. 26—shows the pore size distribution obtained from the adsorption curve of the isotherm for a AgNP doped 8 wt % cellulose nanoparticle aerogel of the present invention produced using the procedure 2;

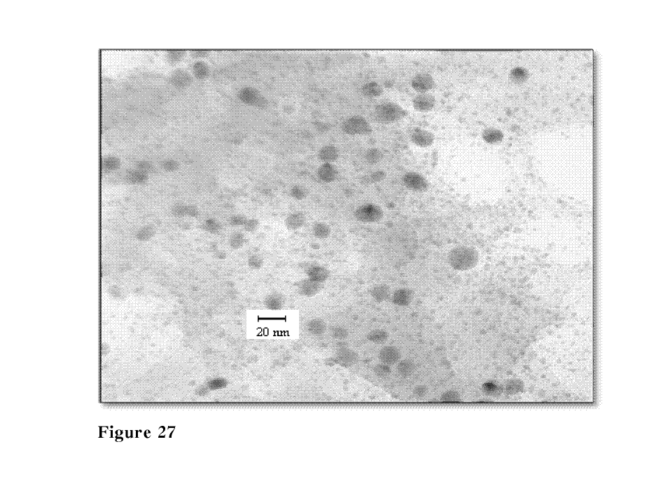

FIG. 27—shows a TEM image of a AgNP doped 8 wt % cellulose nanoparticle aerogel of the present invention;

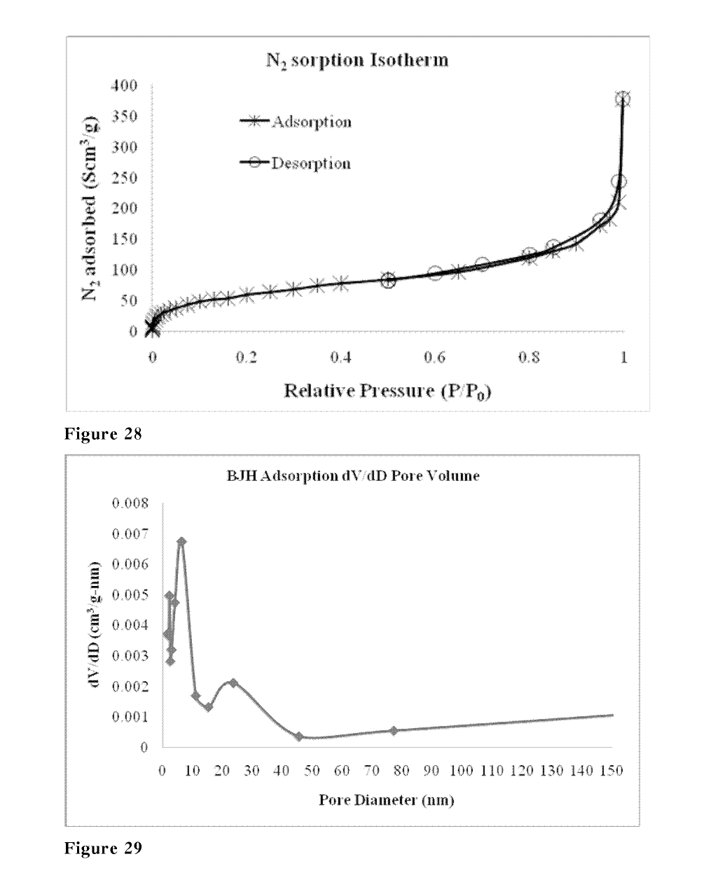

FIG. 28—shows a nitrogen sorption isotherm for a PtNP doped 8 wt % cellulose nanoparticle aerogel of the present invention produced using the procedure 2;

FIG. 29—shows the pore size distribution obtained from the adsorption curve of the isotherm for a PtNP doped 8 wt % cellulose nanoparticle aerogel of the present invention produced using the procedure 2;

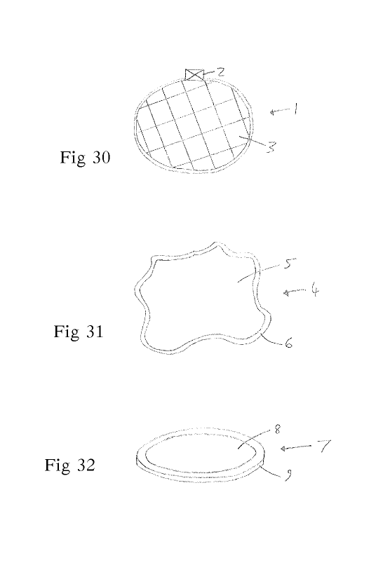

FIG. 30—shows a cross section of a spherical steel H₂ container according to the invention containing a cellulose nanoparticle aerogel according to the invention;

FIG. 31—shows a cross section of a renewable porous catalyst support according to the invention;

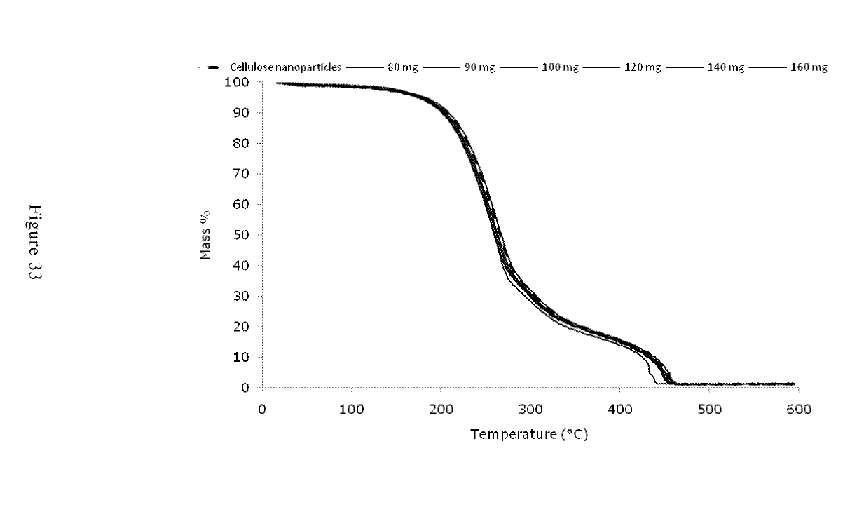

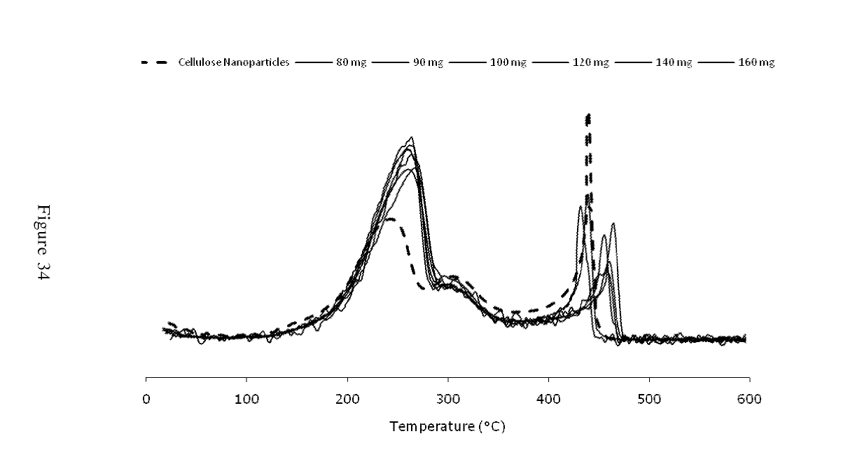

FIG. 32—shows a particle filter according to the invention;

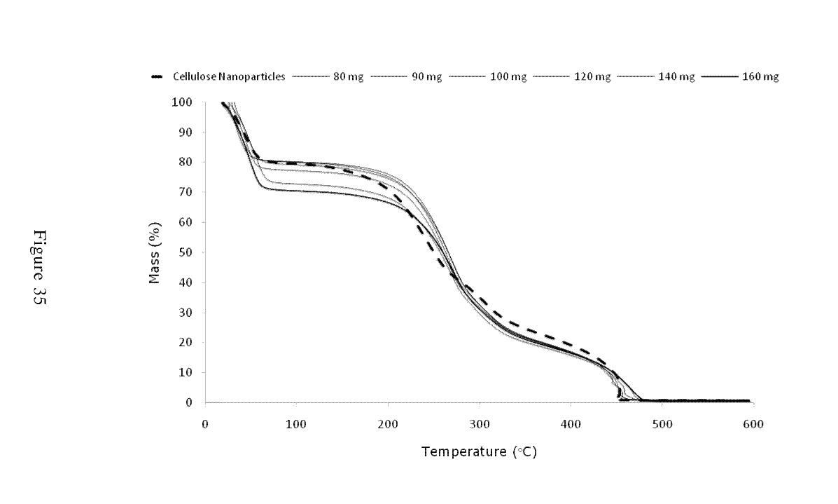

FIG. 33—shows TGA curves of cellulose nanoparticles and six aerogel samples of various initial cellulose nanoparticle mass at 0% relative humidity;

FIG. 34—shows DTG profiles of cellulose nanoparticles and six aerogel samples of various initial cellulose nanoparticle mass at 0% relative humidity;

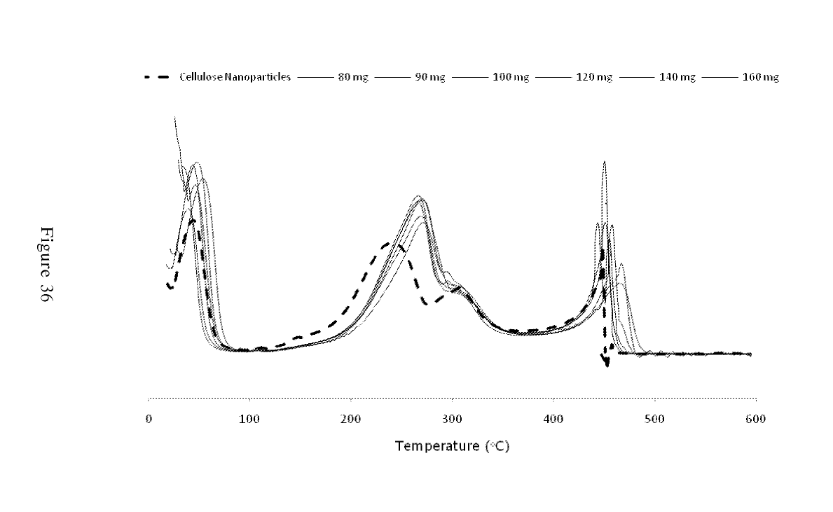

FIG. 35—shows TGA curves of cellulose nanoparticles and six aerogel samples of various initial cellulose nanoparticle mass at 98% relative humidity; and

FIG. 36—shows DTG profiles of cellulose nanoparticles and six aerogel samples of various initial cellulose nanoparticle mass at 98% relative humidity.

FIG. 7 shows the different types of pores which may be present within a porous material. The pores are classified with respect to their availability to an external fluid. Pores (b) and (f) are classified as blind pores as they are only open at one end. Pore (e) is classified as a through pore as it is open at both ends. Pores (c) and (f) are cylindrical, pore (b) is ink-bottle shaped and pore (d) is funnel shaped.

### RESULTS AND DISCUSSION

The cellulose nanoparticle aerogels of the present invention act in a similar way to previously reported cellulose aerogels and act as suitable host materials for the incorporation of different species. The applications of the cellulose nanoparticle aerogels are extended with the incorporation of different species into the aerogel network. The application potential for the cellulose nanoparticle aerogels is, therefore, also increased by the incorporation of unique species. Electrically conducting cellulose nanoparticle aerogels have been prepared by incorporating carbon nanotubes into their structure. Additionally, we have incorporated metal nanoparticles into the cellulose nanoparticle aerogel structure to produce and characterize materials which are suitable for e.g. electro-optical, antibacterial and catalytic applications. Cellulose hydrogels and organogels have also been prepared.

### Example 1

### Cellulose Nanoparticle Aerogels

Cellulose nanoparticles were first prepared by the acid hydrolysis of cotton as described in the following Experimental Procedures section under heading “preparation of cellulose nanocrystals”. Other cellulose sources can alternatively be used. These cellulose nanoparticles were then used to produce aerogels in a three step method as described in the Experimental Procedures section. The first step involved the production of a hydrogel using sonication. The second step involved the hydrogel being subjected to solvent exchange in an excess of anhydrous ethanol at 25° C. to form an organogel. This organogel was then dried under continuous flow of scCO₂ at a temperature of 40° C. and a pressure of 10 000 kPa (100 bar) to yield an aerogel. The concentration of cellulose nanoparticles used in the formation of each aerogel is expressed as wt %, rounded to the nearest integer. Wt % is expression of the amount of cellulose in water-cellulose mixture in the starting hydrogel (e.g. 8 wt % is 80 mg cellulose nanoparticles in 1 ml of water). The aerogels were all characterized using density and porosity calculations, X-ray diffraction and BET nitrogen sorption.

The density of the aerogels was calculated simply by dividing their mass by their volume. The volume was taken to be an average of five measurements carried out on the aerogel. The obtained density range for aerogels with cellulose nanoparticle concentrations between 8 and 16 wt % was 0.0721 to 0.1447 g/cm³. FIG. 9 shows that the density of the aerogels is dependent on the initial cellulose nanoparticle concentration. The density of the aerogel increases with an increasing cellulose nanoparticle concentration.

The percentage porosity of the aerogels was calculated using the previously determined density values and the density of the bulk cellulose nanoparticles (1.59 g/cm³) using Equation 2.

$$P = ( 1 - \frac{{d}_{p}}{{d}_{b}} ) \times 100$$

where dₚ is the density of the porosity material. d_{b} is the density of the bulk cellulose nanocrystals, 1.59 g/cm³.

FIG. 10 shows that the percentage porosity of the aerogels depends on the cellulose nanoparticle concentration. As the concentration of the cellulose nanoparticles increases, the percentage porosity decreases. The porosity of the aerogels lies in the range of 91-96%.

X-ray powder diffraction (XRD) can be used to ensure that the crystallinity of the cellulose is retained in the aerogel samples. FIG. 11 shows the diffractograms obtained for each sample. The diffractograms all show good agreement with each other which indicates that the crystallinity of the cellulose is retained.

The relative crystallinity index (RCI) calculated for the cellulose nanocrystals gave a value of 89.2%. FIG. 12 shows that the RCI values calculated for the aerogel samples vary very little from this value. It can, therefore, be concluded that the crystalline structure of cellulose is largely unaffected by the aerogel production procedure.

BET N₂ sorption measurements were used to provide values for the BET internal surface area, the pore size distribution and the average pore size. FIG. 13 shows the BET surface area values obtained for aerogel samples with increasing initial cellulose nanoparticle concentrations. The values are generally from about 200 m²/g to about 600 m²/g, on average they are around 420 m²/g. Generally, the aerogels produced in this work display internal surface areas in the range of 216 m²/g to 605 m²/g.

FIG. 14 shows the average pore diameter calculated by BET for the aerogel samples. The average pore size remains fairly consistent with increasing cellulose nanoparticle concentration, however, it is expected that the pore size will decrease with increasing cellulose nanoparticle concentration.

The nitrogen sorption isotherm for the 8 wt % sample at 77 K (FIG. 15) shows that the adsorption and desorption curves do not coincide and therefore display adsorption hysteresis. The shape of the isotherm is very similar to the isotherms obtained for all of the cellulose nanoparticle aerogels. The isotherms can be classified as type IV isotherms according to IUPAC recommendations because of their shape and absorption hysteresis. A type IV isotherm indicates that the aerogel is a mesoporous adsorbent. A mesoporous material is characterised by the large quantity of pores in the 2-50 nm diameter size range.

This is confirmed by the pore size distribution (FIG. 16) for the same sample which shows that the majority of the pores occupying the aerogel's volume are in the mesoporous range (2-50 nm). The pore size distribution is calculated from the Kelvin equation using the BJH (Barrett, Joyner and Halenda) method. The BJH method assumes that all pores are cylindrical and can therefore only be used as a rough estimate as the pore structure is likely to be much more complex. FIG. 16 shows that the aerogel displays a fairly wide pore size distribution with the majority of pores displaying diameters in the 10 nm region. The larger pores present within the sample will provide channels and good access to the smaller pores of which the aerogel appears to contain in a significant amount. FIG. 16 is also representative of the pore size distributions observed for all other aerogels.

### Example 2

### Carbon Nanotube Containing Cellulose Nanoparticle Aerogels

The carbon nanotube doped aerogels were prepared using a very similar method to the cellulose nanoparticle aerogels. Water soluble Multi-Walled Carbon NanoTubes (MWNT) were prepared using the procedure described in the Experimental section (either by nitric acid oxidation or gas-phase oxidation) and were then used in the preparation of carbon nanotube containing cellulose nanoparticle aerogels by following the general procedure set out in the Experimental Procedures section under the heading “preparation of carbon nanotube containing cellulose nanoparticle aerogels”. Aerogel samples were characterized using density and porosity calculations, BET nitrogen sorption and conductivity measurements.

Aerogels of 8, 9 and 10 wt % initial cellulose concentration were doped with 1 and 2 wt % carbon nanotubes. FIG. 17 shows that as the cellulose nanoparticle concentration and carbon nanotube concentration increase the density of the aerogel also increases.

The percentage porosity of the aerogels doped with carbon nanotubes is shown in FIG. 18. The porosity of the aerogel decreases with an increasing cellulose nanoparticle and carbon nanotube concentration.

The nitrogen sorption isotherm for the 9 wt % sample doped with 1 wt % carbon nanotubes at 77 K (FIG. 19) shows a similar shape to the isotherm obtained for the cellulose nanoparticle aerogels. The adsorption and desorption curves do not coincide and therefore display adsorption hysteresis. This isotherm can be classified as a type IV isotherm, indicating that the aerogel retains its mesoporous structure. The BET surface area of the 8 wt % and 10 wt % cellulose nanoparticles aerogels containing 1 wt % carbon nanotubes was determined to be 156 m² g, 418 m²/g and 285 m²/g respectively.

The pore size distribution (FIG. 20) for the same sample confirms that the majority of the pores occupying the aerogel's volume are in the mesoporous range (2-50 nm). The aerogel displays a fairly wide pore size distribution with the majority of pores displaying diameters in the 10 nm region. The pore size distribution is also very similar to the pore size distribution obtained for the 9 wt % cellulose nanoparticle aerogel.

Various conductivity measurements were conducted on aerogel samples having 9 wt % cellulose nanoparticles doped with 1 wt % carbon nanotubes, 10 wt % cellulose nanoparticles doped with 1 wt % carbon nanotubes and 10 wt % cellulose nanoparticles doped with 2 wt % carbon nanotubes.

FIG. 21 shows the typical plots achieved by scanning the potential from +50 V to −50 V and back again and plotting the current against the voltage.

The conductance was measured from the slope of the linear fit to the data and was then converted into a conductivity measurement using the Equation below.

$$σ = \frac{G \times sample length}{sample cross sectional area}$$

The equation used to calculate the conductivity in Scm⁻¹.

The conductivity values obtained for the cellulose nanoparticle aerogels doped with carbon nanotubes are displayed in Table 1.

TABLE 1
Example values obtained for the conductivity of the carbon
nanotube doped cellulose nanoparticle aerogels.

| | Conductivity measurements (S cm⁻¹) | |
|---|---|---|
| | Aerogel containing | Aerogel containing |
| | 1 wt % carbon | 2 wt % carbon |
| | nanotubes | nanotubes |
| Aerogel made with 9 wt % | 5.2325 × 10⁻¹⁰ | |
| cellulose nanoparticles | | |
| Aerogel made with 10 wt % | 2.6988 × 10⁻¹⁰ | 5.2325 × 10⁻¹⁰ |
| cellulose nanoparticles | | |

### Example 3

### Metal Nanoparticle Containing Cellulose Nanoparticle Aerogels

### Gold Containing Cellulose Nanoparticle Aerogels

The first metal nanoparticle that was tried for its ease of insertion into the cellulose nanoparticle aerogels was gold.

In-situ reduction of metal ions can be carried out by adding the metal salt solutions to cellulose hydrogels. It is thought that cellulose acts as the reducing agent for the metal salts and the reducing end groups and hydroxyl groups on the cellulose reduce the metal ions from the salt solutions. Cellulose nanoparticle hydrogels can reduce the metal centre in metal salts without the need for additional reducing agents such as NaBH, due to the large quantity of hydroxyl groups on the nanoparticle surface. However, NaBH₄ can be added to provide additional reduction power.

Gold containing cellulose nanoparticle aerogels were made following Procedure 2 under the heading “Preparation of Gold containing cellulose nanoparticle aerogels” from the Experimental procedures section. An 8 wt % cellulose nanoparticle hydrogel was used as the reaction medium. The HAuCl₄ was added to the hydrogel and was left at 25° C. for 24 hours. As the hydrogel was not subjected to elevated temperatures the cellulose nanoparticles remained intact. The hydrogel containing AuNPs was then solvent exchanged and dried under supercritical conditions as described in the above method. This aerogel was subsequently characterised using density and porosity calculations, BET N₂ sorption measurements (shown in Table 2) and Environmental Scanning Electron Microscopy (ESEM).

TABLE 2
Comparison of the values obtained for the 8 wt % cellulose
nanoparticle aerogel and the AuNP doped 8 wt % cellulose
nanoparticle aerogel.

| | | 8 wt % cellulose |
|---|---|---|
| | 8 wt % cellulose | nanoparticle aerogel |
| | nanoparticle aerogel | doped with AuNPs |
| Density (g/cm³) | 0.0721 | 0.0799 |
| Porosity (%) | 96 | 95 |
| BET surface area (m²/g) | 524.869 | 392 |

The density of the aerogel increases when it is doped with AuNPs and the percentage porosity decreases. The BET surface area is reduced with the incorporation of AuNPs into the porous aerogel network. This is due to the increase in the weight with the addition of gold but can also be due to some extent to the blocking of some pores with AuNPs and/or larger Au structures.

The sharp adsorption curve on the N₂ sorption isotherm (FIG. 22) indicates that the pore size will continue to increase into the macropore region. This observation is confirmed by the pore size distribution (FIG. 23) which shows that there is a wide range of pore sizes within the aerogel.

FIG. 24 shows the ESEM images of the AuNP doped aerogel. FIG. 24a shows the highly porous internal network of the aerogel and FIG. 24b shows a typical AuNP stabilized within the porous cellulose nanoparticle network.

### Example 4

### Silver Nanoparticle Containing Cellulose Nanoparticle Aerogels

Two different production methods were used to reduce AgNO₃, to produce aerogels containing silver nanoparticles (AgNPs), these are Procedures 1 and 2 of the “Preparation of silver nanoparticle containing cellulose nanoparticle aerogels” part of the “Experimental Procedures” section. The aerogels were characterized using density and porosity calculations, BET N₂ sorption measurements (shown in Table 3), X-ray Photoelectron Spectroscopy (XPS) and Transmission Electron Microscopy (TEM).

TABLE 3
Comparison of the values obtained for the 8 wt % cellulose
nanoparticle aerogel and two AgNP doped 8 wt % cellulose
nanoparticle aerogels.

| | 8 wt % | 8 wt % cellulose | 8 wt % cellulose |
|---|---|---|---|
| | cellulose | nanoparticle aerogel | nanoparticle aerogel |
| | nanoparticle | doped with AgNP- | doped with AgNP- |
| | aerogel | procedure 1 | procedure 2 |
| Density | 0.0721 | 0.0991 | 0.1197 |
| (g/cm³) | | | |
| Porosity (%) | 96 | 94 | 93 |
| BET surface | 524.869 | 207 | 185 |
| area (m²/g) | | | |

The AgNP doped aerogels produced by both methods display a higher density and lower percentage porosity than the original cellulose nanoparticle aerogel. The BET surface area decreases with the loading of AgNPs as the weight of the aerogel increases with silver addition and some pores may become blocked with AgNPs and/or larger Ag structures.

The shape of the N₂ sorption isotherm (FIG. 25) indicates that the open mesoporous structure is retained in the AgNP doped aerogels using both procedures.

The pore size distribution (FIG. 26) indicates that the aerogels have a large pore size range but also a significant amount of pores in the 10 nm region for both procedures.

The presence of Ag (0) on the surface of the aerogels doped with AgNP has been confirmed using XPS. Table 4 shows that the values obtained for Ag 3d_{5/2} and Ag 3d_{3/2} have been shifted slightly to lower values compared to the signal for Ag(I) in AgNO3 for both of the AgNP doped aerogels. The energy values obtained for Ag 3d_{5/2} in both aerogel samples show good agreement with values previously reported for metallic Ag of 368.1 eV. It can be concluded therefore that the Ag⁺ ions are reduced to metallic Ag in the hydrogels by the cellulose nanoparticles.

TABLE 4
The binding energies of Ag 3d_{5/2} and Ag 3d_{3/2} for the two AgNP
doped aerogels.

| | Binding energies determined by XPS | |
|---|---|---|
| | 8 wt % cellulose | 8 wt % cellulose |
| | nanoparticle aerogel doped | nanoparticle aerogel doped |
| | with AgNP-procedure 1 | with AgNP-procedure 2 |
| Ag 3d_{5/2} (eV) | 368.26 | 368.28 |
| Ag 3d_{3/2} (eV) | 374.26 | 374.29 |

Table 5 shows that the aerogel made using the second procedure contains the highest wt % of Ag on its surface. This is because the AgNO₃ was sonicated with the cellulose nanoparticles and so the Ag⁺ ions would have been dispersed more efficiently. The Ag⁺ ions would then have better access to the reducing groups on the surface of the nanoparticles, allowing them to be more efficiently reduced. In the first procedure the AgNO₃ was not sonicated with the cellulose nanoparticles and so the silver ions would have had a more limited access to the cellulose reducing groups (due to diffusion effects). The silver loading content seems to improve on previously reported values for silver/polymer nanocomposites of 0.50 wt % of Ag and 1.00 wt % of Ag.

TABLE 5
The elemental content of the silver nanoparticle doped aerogels
given in wt % using XPS.

| | | 8 wt % cellulose |
|---|---|---|
| | | nanoparticle |
| | | aerogel doped |
| | 8 wt % cellulose nanoparticle | with silver |
| | aerogel doped with silver | nanoparticles- |
| | nanoparticles-procedure 1 | procedure 2 |
| Silver content (wt %) | 1.80 | 2.49 |

The silver nanoparticle containing aerogel was also examined using TEM. FIG. 27 shows that the silver nanoparticles are dispersed well throughout the aerogel structure. The presence of silver in the aerogel structure was confirmed by Energy Dispersive X-ray (EDX) analysis.

### Example 5

### Platinum Nanoparticle Containing Cellulose Nanoparticle Aerogels

Two different production methods were used to reduce H₂PtCl₆ to produce aerogels containing platinum nanoparticles (PtNPs). The methods used were procedures 1 and 2 of “Preparation of platinum nanoparticle containing cellulose nanoparticle aerogels” from the “Experimental procedures” section. The aerogels were characterized using density and porosity calculations and BET N₂ sorption measurements (as shown in Table 6).

TABLE 6
Comparison of the values obtained for the 8 wt % cellulose
nanoparticle aerogel and two PtNP doped 8 wt % cellulose nanoparticle
aerogels.

| | | | 8 wt % cellulose |
|---|---|---|---|
| | 8 wt % | 8 wt % cellulose | nanoparticle |
| | cellulose | nanoparticle aerogel | aerogel |
| | nanoparticle | doped with PtNP- | doped with PtNP- |
| | aerogel | procedure 1 | procedure 2 |
| Density (g/cm³) | 0.0721 | 0.0795 | 0.0899 |
| Porosity (%) | 96 | 95 | 94 |
| BET surface area | 524.869 | 461.5 | 175 |
| (m²/g) | | | |

As expected, the density of the aerogels increases with the incorporation of PtNPs and the percentage porosity decreases. The BET surface area decreases with the loading of platinum nanoparticles due to the increase in weight of the gel with platinum addition but also potentially due to platinum nanoparticles and/or larger Pt structures blocking the pores of the aerogel.

The N₂ sorption isotherm (FIG. 28) is of a similar shape to the 8 wt % cellulose nanoparticle aerogel and so it can be concluded that the mesoporous structure is retained within the PtNP doped aerogels.

The pore size distribution (FIG. 29) shows that there is a wide range of pore diameters within the aerogels but also that there is a significant amount of pores with diameters in the 10 nm region.

### Experimental Procedures

### General

All samples were weighed using a Mettler Toledo AB265-S/Fact (±0.01 mg). The measurements of gel dimensions were conducted using a vernier calliper (±0.02 mm). Measurements of the dimensions were an average of five measurements. Hydrogels were made and contained in glass vials with an average internal diameter of 10.0 mm and an average length of 46.4 mm. Centrifugation of the cellulose nanocrystals was conducted in a Sigma Laboratory Refrigerated Centrifuge 6K15 (10 000 rpm, 10° C.). Sonication of the cellulose nanoparticles was completed using a Branson digital sonifier (5 minutes, in three second pulses with two second intervals, an amplitude of 15%, maximum temperature 35° C.). The cellulose nanoparticle suspension was freeze dried using a Heto PowerDry LL3000 Freeze Dryer. Sonication of the cellulose nanoparticles to obtain hydrogels was completed in a Sonomatic 375 Ultrasonic Cleaner, Agar Scientific (maximum temperature 25° C.). Centrifugation of the platinum nanoparticle, cellulose nanoparticle and deionised water dispersion was conducted using an Eppendorf Minispin (10 000 rpm).

### Materials

Cotton wool was supplied by Boots and was used as received. Sulfuric acid was supplied by Fischer, >95% purity and was used as a 64 wt % aqueous solution. Amberlite MB 6113 was supplied by Fluka. Carbon dioxide (purity 99.9%) was supplied by Cryoservice. MWNT were received from Nanocyl (#3100), average diameter 10 nm. Anhydrous ethanol and silver nitrate (AgNO₃) were received from Sigma Aldrich. Hydrochloric acid, ethanol, polytetrafluoroethylene (PTFE) membrane (0.45 μm pore diameter), nitric acid, tetrachloroauric acid trihydrate (HAuCl₄.3H₂O), trisodium citrate dihydrate, tannic acid, copper sulfate and chloroplatinic acid hydrate (H₂PtCl₆.xH₂0) were all received from Aldrich. Deionised water was used in all experiments.

### Analysis Procedures and Instrumentation

Densities of the samples were determined simply by weighing and measuring their dimensions. Porosities of the samples were calculated using the densities of the samples and the density of the bulk cellulose nanoparticles (1.59 g/cm³). XRD measurements were carried out on the aerogel samples using a Phillips X′ pert PW 3710 diffractometer (CuK_{α} radiation, wavelength 1.5406 Å). The scans were performed with a 2θ increment of 0.02° between 5° and 40°. BET analysis of all samples was run on a Micrometrics ASAP 2000. N₂ adsorption and desorption at 77K was utilized to determine the specific surface areas and porosities of the prepared aerogels. From the obtained isotherms the BET-surface and the BJH-pore size distribution were calculated. Electron micrographs of the samples were obtained using environmental scanning electron microscopy (ESEM) Instrument FEI XL30 FEG ESEM with energy dispersive X-ray (EDX) analysis. Conductivity Measurements were conducted on the carbon nanotube containing aerogels using a Keithley Series 2400 SourceMeter Instrument, scanning the potential from −50 V to 50 V. XPS analysis was conducted on the silver nanoparticle cellulose nanoparticle aerogels using a Kratos AXIS Ultra spectrometer. The photoelectron spectrometer was equipped with a monochromated Al K_{α} X-ray source (1486.6 eV, 10 kV, 15 mA, 150 W). Survey and high resolution spectra were recorded at pass energies of 80 and 20 eV, respectively. TEM analysis was carried out on the AuNP cellulose nanoparticle aerogel using a Jeol JEM-2000 FX II high resolution TEM.

### Preparation of Cellulose Nanocrystals

### General Procedure

Cellulose nanocrystals were obtained by the acid hydrolysis of cotton wool for 35 minutes at 45° C. in a 64 wt % aqueous H₂SO₄ solution whilst agitating constantly using a mechanical stirrer. The cellulose nanocrystals were then washed with distilled water and centrifuged using a Sigma Laboratory Refrigerated Centrifuge 6K15 (10 000 rpm, 10° C.) for 20 minutes. The first centrifugation was followed by the redispersion of the cellulose nanocrystals in distilled water and another centrifugation for 30 minutes. This process was repeated a third time, however the third centrifugation lasted for 40 minutes. Dialysis of the nanocrystals was then used to remove any remaining free acid in the dispersion. The nanocrystals were dialysed using dialysis bags and constantly flowing tap water for 48 hours. Subsequently, sonication of the washed and dialysed nanocrystals dispersed the particles. The sonication was completed using a Branson digital sonifier for 5 minutes, in three second pulses with two second intervals at an amplitude of 15% with a maximum temperature of 35° C. Filtering the solution under suction then removed any remaining non-hydrolysed cellulose fibres. Amberlite MB, a solid ion exchanger, was then added, under agitation using a magnetic stirrer for one hour, to the solution to protonate the surface of the cellulose nanocrystals. The Amberlite was removed from the solution using suction filtration. The suspension was redispersed using the sonifier for approximately two minutes. After the final sonication, the suspension was plunged into liquid nitrogen to freeze before being attached to the Heto PowerDry LL3000 Freeze Dryer until it was completely dry.

### Preparation of Cellulose Nanoparticle Hydrogels

### General Procedure

Cellulose nanoparticles were dispersed in deionised water in a small glass vial. The resultant dispersion was sonicated using a sonication bath (Sonomatic 375 Ultrasonic Cleaner, Agar Scientific) at a maximum temperature of 25° C. for 30-60 minutes. The duration of the sonication was dependent upon the concentration of the cellulose nanoparticles. The sonication time decreases for a higher initial cellulose nanoparticle concentration (Table 7). All concentrations are expressed as weight percentages (wt %); these wt % are rounded to the nearest whole number.

### Quantities

TABLE 7
The quantities of cellulose nanoparticles used to produce hydrogels
of varying concentrations (wt %).

| | Cellulose | Deionised | Sonication |
|---|---|---|---|
| | nanoparticles | water | time |
| Wt % | (mg) | (ml) | (mins) |
| 8 | 80 | 1 | 60 |
| 9 | 90 | 1 | 60 |
| 10 | 100 | 1 | 60 |
| 12 | 120 | 1 | 45 |
| 14 | 140 | 1 | 45 |
| 16 | 160 | 1 | 30 |

In addition to the above results, we have also successfully scaled up the process to use 100 ml of deionised water in the production of hydrogels.

### Preparation of Cellulose Nanoparticle Organogels

### General Procedure

The hydrogels in the glass vial moulds were placed into anhydrous ethanol overnight. The water in the pores of the hydrogel was replaced with the anhydrous ethanol by solvent exchange. The following day the glass vial mould was cracked using a diamond pen and weighed, and the gel was re-immersed in new anhydrous ethanol. The replacing of the anhydrous ethanol over a period of four days acted as an aging procedure to stabilize the network structure within the gel and ensured that the solvent exchange process was complete.

### Preparation of Cellulose Nanoparticle Aerogels

### General Procedure

Cellulose nanoparticle organogels were removed from their glass vial moulds and were weighed. The organogels were dried using supercritical CO₂ in the apparatus detailed as follows. The organogels were placed into a high pressure stainless steel autoclave on a polytetrafluoroethylene (PTFE) support and were covered with anhydrous ethanol (˜2.5 ml) to prevent premature drying of the organogel by solvent evaporation. The autoclave was attached inside the oven and was heated to 40° C. as carbon dioxide was pumped into the autoclave to a pressure of 10 000 kPa (100 bar). The pressure inside the autoclave reached 10 000 kPa (100 bar) after 5 minutes. The flow of carbon dioxide was kept at a constant 2 ml/min until the anhydrous ethanol was completely removed (˜6 hours). The autoclave was subsequently depressurized by 500 kPa/min (5 bar/min) over 20 minutes. The dry aerogel was then removed from the autoclave and was weighed.

Shortened Carbon Nanotubes by Gas-Phase Oxidation (MWNT_{60% shortened})

### General Procedure

MWNT (100 mg, as-received) were placed into an alumina crucible and annealed in air at 550° C. and the decrease in mass recorded. The obtained solids were then sonicated in concentrated hydrochloric acid (37% HCl, 50 ml) at 25° C. for 30 minutes. The subsequent solution was diluted with deionised water (250 ml), filtered with a PTFE membrane under vacuum, rinsed thoroughly with deionised water and ethanol and sucked dry. Residual water was removed by drying under vacuum to yield a black solid (˜40 mg).

### Oxidation of Nanotubes in Nitric Acid MWNT-COOH)

### General Procedure

The annealed MWNT (25 mg) were sonicated in nitric acid (2.6 M HNO₃, 50 mL) at 25° C. for 15 minutes and then refluxed at 120° C. for 48 hours. The obtained black suspension was diluted with deionised water (100 ml), filtered with a PTFE membrane under vacuum, rinsed thoroughly with deionised water and ethanol and finally sucked dry. Any residual water was removed by drying under vacuum to yield a black solid (23.67 mg, 94.6%).

### Preparation of Carbon Nanotube Containing Cellulose Nanoparticle Aerogels

### General Procedure

MWNT were dispersed in deionised water in a small vial and sonicated for 15 minutes in a sonication bath (Sonomatic 375 Ultrasonic Cleaner, Agar Scientific). Cellulose nanoparticles were then added to the dispersed MWNT. The resultant dispersion was further sonicated at a maximum temperature of 25° C. for 60 minutes. All concentrations are expressed as weight percentages (wt %); these weight percentages are rounded up to the nearest whole number.

TABLE 8
The quantities of cellulose nanoparticles and carbon nanotubes
used to produce aerogels of varying concentrations (wt %).

| | Cellulose | Deionised | Carbon | Sonication |
|---|---|---|---|---|
| | nanoparticles | water | nanotubes | time |
| Wt % | (mg) | (ml) | (mg) | (minutes) |
| 8 | 80 | 1 | 1 | 60 |
| 8 | 80 | 1 | 2 | 60 |
| 9 | 90 | 1 | 1 | 60 |
| 9 | 90 | 1 | 2 | 60 |
| 10 | 100 | 1 | 1 | 60 |
| 10 | 100 | 1 | 2 | 60 |

The hydrogels in the glass vial moulds were placed into anhydrous ethanol overnight. The following day the glass vial mould was cracked using a diamond pen and weighed, and the gel was re-immersed in new anhydrous ethanol. The replacing of the anhydrous ethanol over a period of four days acted as an aging procedure to stabilize the network structure within the gel and ensured that the solvent exchange process had completed. The MWNT cellulose nanoparticle organogels were then removed from their glass vial moulds and were weighed. The organogels were dried using supercritical CO₂ in the apparatus detailed previously.

### Preparation of Gold Nanoparticle Containing Cellulose Nanoparticle Aerogels

An 8 wt % hydrogel was first made following the general procedure with cellulose nanoparticles (80 mg) being sonicated in deionised water (1 ml) for 60 minutes. Tetrachloroauric acid trihydrate, HAuCl₄.3H₂O (16.99 mg), was subsequently added to the hydrogel. The hydrogel was then left at 25° C. for 4 days. The hydrogel was subsequently placed into anhydrous ethanol overnight. The following day the glass vial mould was cracked using a diamond pen and weighed, and the gel was re-immersed in new anhydrous ethanol. The anhydrous ethanol was replaced every day for a period of four days. The AuNP containing cellulose nanoparticle organogel was then removed from the glass vial mould and weighed. The organogel was dried using supercritical CO₂ in the rig described previously to yield an aerogel.

### Preparation of Silver Nanoparticle Containing Cellulose Nanoparticle Aerogels

### Procedure 1

An 8 wt % hydrogel was first made following the general procedure with cellulose nanoparticles (80 mg) being sonicated in deionised water (1 ml) for 60 minutes. The silver metal salt, AgNO₃ (8.49 mg), was subsequently added to the hydrogel. The mixture was then put into a sealed vessel containing hydrated copper sulfate (saturated solution), CuSO₄. The CuSO₄ acted to produce an atmosphere within the sealed vessel of 98% humidity at room temperature. The humid atmosphere stopped water from the hydrogel being removed at a temperature of 80° C. The sealed vessel containing the hydrogel was put into the oven at 80° C. for 24 hours. The hydrogel was subsequently placed into anhydrous ethanol overnight. The following day the glass vial mould was cracked using a diamond pen and weighed, and the gel was re-immersed in new anhydrous ethanol. The anhydrous ethanol was replaced every day for a period of four days. The silver nanoparticle containing cellulose nanoparticle organogel was then removed from the glass vial mould and weighed. The organogel was dried using supercritical CO₂ in the rig described previously to yield an aerogel.

### Procedure 2

Cellulose nanoparticles (80 mg) and the silver metal salt, AgNO, (8.49 mg), were sonicated in deionised water (1 ml) for 60 minutes until a firm hydrogel was created. The hydrogel was subsequently put into a sealed vessel containing hydrated copper sulphate (saturated solution), CuSO₄. The CuSO₄ acted to produce an atmosphere within the sealed vessel of 98% humidity at room temperature. The humid atmosphere stopped water from the hydrogel being removed at a temperature of 80° C. The sealed vessel was then put into the oven at 80° C. for 24 hours. The hydrogel was subsequently placed into anhydrous ethanol overnight. The following day the glass vial mould was cracked using a diamond pen and weighed, and the gel was re-immersed in new anhydrous ethanol. The anhydrous ethanol was replaced every day for a period of four days. The silver nanoparticle containing cellulose nanoparticle organogel was then removed from the glass vial mould and weighed. The organogel was dried using supercritical CO₂ in the rig described previously to yield an aerogel.

### Preparation of Platinum Nanoparticle Containing Cellulose Nanoparticle Aerogels

### Procedure 1

Cellulose nanoparticles (80 mg), H₂PtCl₆.xH₂O (20.49 mg) and deionised water (1 ml) were put in a vial placed into a sealed vessel containing hydrated copper sulphate (saturated solution), CuSO₄. The CuSO₄ acted to produce an atmosphere within the sealed vessel of 98% humidity at room temperature to stop the evaporation of water from the suspension. The sealed vessel was then put into the oven at 80° C. for 24 hours. The resulting suspension was centrifuged at 10 rpm for 10 minutes. The liquid was removed from the sample and was replaced with new deionised water (1 ml). The suspension was then centrifuged again at 10 rpm for 10 minutes. This process was repeated a further 3 times. The final liquid washing was removed and deionised water (1 ml) was added to yield a platinum nanoparticle and cellulose nanoparticle suspension. This suspension was then sonicated for 60 minutes. The hydrogel was subsequently placed into anhydrous ethanol overnight. The following day the glass vial mould was cracked using a diamond pen and weighed, and the gel was re-immersed in new anhydrous ethanol. The anhydrous ethanol was replaced every day for a period of four days. The platinum nanoparticle containing cellulose nanoparticle organogel was then removed from the glass vial mould and weighed. The organogel was dried using supercritical CO₂ in the rig described previously to yield an aerogel.

### Procedure 2

An 8 wt % hydrogel was first made following the general procedure with cellulose nanoparticles (80 mg) being sonicated in deionised water (1 ml) for 60 minutes. H₂PtCl₆.xH₂0 (20.49 mg) was subsequently added to the hydrogel. The hydrogel was then left at 25° C. for 24 hours. The hydrogel was subsequently placed into anhydrous ethanol overnight. The following day the glass vial mould was cracked using a diamond pen and weighed, and the gel was re-immersed in new anhydrous ethanol. The anhydrous ethanol was replaced every day for a period of four days. The platinum nanoparticle containing cellulose nanoparticle organogel was then removed from the glass vial mould and weighed. The organogel was dried using supercritical CO₂ in the rig described previously to yield an aerogel.

### Uses of the Invention

FIG. 30 shows a cross section of a spherical steel H₂ container 1 according to the invention comprising a mechanical valve 2. Container 1 contains a cellulose nanoparticle aerogel 3 according to the invention. In use, H₂ can be introduced into cellulose nanoparticle aerogel 3 of container 1 via valve 2 under a pressure of for instance 3×10⁶ Pa. The hysteretic properties of the cellulose nanoparticle aerogel 3 control the release rate of the H₂ andallow it to be stored at lower pressure and closer to ambient temperatures than it would ordinarily.

FIG. 31 shows a cross section of a renewable porous catalyst support 4 according to the invention. The renewable porous catalyst support 4 comprises a cellulose nanoparticle aerogel 5 with metallic groups 6 or metal nanoparticles, metal containing nanoparticles, or metal alloy nanoparticles immobilised on the aerogel. The metallic groups supported on the renewable porous catalyst supports act as catalysts in a similar fashion to the metal centres in zeolites.

FIG. 32 shows a particle filter 7 according to the invention. A layer of cellulose nanoparticle aerogel 8 is held in casing 9. The filter 7 is in this embodiment an air purifying filter but the aerogel 8 can be fine tuned to filter out specific particles as desired.

We were interested in establishing if a sonication process, or the supercritical drying process used in providing an aerogel altered the cellulose particles. Thermal gravimetric analysis (TGA) was used to determine whether the sonication or supercritical drying processes altered the thermal stability of the cellulose nanoparticle aerogels in comparison to the starting material. TGA analyses the change in weight of the aerogels as a function of increasing temperature. The TGA curves obtained from the thermal treatment of cellulose nanoparticles and cellulose nanoparticle aerogels in air are shown in FIG. 33. The TGA curves for the aerogels are all extremely similar. The corresponding differential thermal gravimetric (DTG) profiles for the cellulose nanoparticles and the aerogels are shown in FIG. 34. The DTG profiles for the aerogels of varying initial cellulose nanoparticle content are again extremely similar. FIGS. 35 and 36 show GBA and DTG curves at 98% relative humidity, and again the profiles of the initial cellulose nanoparticles and of the aerogel are strikingly similar.

From looking at the graphs of FIGS. 33 to 36, we would say that the curve for a hydrogel is within 2%, 5%, or 10%, or 15% of the value of the curve for the cellulose nanoparticles themselves for at least 80%, or 90%, or 100% of the temperature range 0° C. to 500° C.

The thermal decomposition patterns of cellulose nanoparticles and cellulose nanoparticle aerogels can be described by two different mechanisms. The first mechanism occurs at temperatures below 300° C. and the second mechanism occurs at temperatures above 300° C. In the first mechanism of cellulose degradation, the molecular weight is reduced by depolymerisation caused by dehydration reactions. The main products of thermal decomposition are water, char residues, carbon monoxide and carbon dioxide. The rate of formation of CO and CO₂ is accelerated in air as the temperature increases. The second mechanism involves the cleavage of secondary bonds and the formation of intermediate products which are then converted into low weight polysaccharides and finally to carbonized products. Cellulose nanoparticles decompose between 106° C. and 473° C., with two maxima at 237° C. and 496° C. The aerogels decompose over a similar range between 112° C.±6.9 (SD) and 467° C.±6.9 (SD) with two maxima at 260° C.±3.1 (SD) and 450° C.±12.5 (SD).

Table 1, below, shows the results obtained from differential thermal analysis (DTA). The onset temperature, degradation temperatures, maximum weight loss rate and percentage weight loss are similar for cellulose nanoparticles and their analogous aerogels indicating that neither the sonication process nor the supercritical drying altered the cellulose nanoparticles chemically.

TABLE 1
Onset temperature, degradation temperature (Tmax), maximum weight-loss rate
(WLRmax), weight loss (WL) in the thermal degradation processes of cellulose
nanoparticles and cellulose nanoparticle aerogels obtained from DTG curves.

| | Relative | Onset | First Process | | | Second Process | | |
|---|---|---|---|---|---|---|---|---|
| | Humidity | temp | Tmax | WLRmax | WL | Tmax | WLRmax | WL |
| Sample | (%) | (° C.) | (° C.) | (% min⁻¹) | (%) | (° C.) | (% min⁻¹) | (%) |
| Cellulose | 0 | 106 | 237 | 2.73 | 67.55 | 439 | 3.02 | 25.23 |
| nanoparticles | 98 | 108 | 235 | 3.13 | 51.32 | 448 | 2.74 | 28.03 |
| 80 mg aerogel | 0 | 106 | 261 | 3.15 | 78.46 | 454 | 1.98 | 15.38 |
| | 98 | 112 | 264 | 2.38 | 59.61 | 468 | 1.92 | 16.46 |
| 90 mg aerogel | 0 | 103 | 262 | 3.26 | 79.88 | 431 | 1.94 | 14.54 |
| | 98 | 105 | 262 | 2.22 | 56.92 | 450 | 1.96 | 15.63 |
| 100 mg aerogel | 0 | 105 | 257 | 3.18 | 80.05 | 455 | 1.76 | 14.61 |
| | 98 | 103 | 266 | 2.28 | 53.58 | 457 | 1.88 | 15.05 |
| 120 mg aerogel | 0 | 106 | 259 | 3.18 | 77.97 | 438 | 2.00 | 17.22 |
| | 98 | 102 | 263 | 2.31 | 59.04 | 450 | 2.01 | 17.75 |
| 140 mg aerogel | 0 | 107 | 256 | 3.18 | 76.92 | 463 | 2.05 | 16.32 |
| | 98 | 109 | 267 | 2.31 | 58.51 | 454 | 1.88 | 17.90 |
| 160 mg aerogel | 0 | 102 | 264 | 3.18 | 78.71 | 458 | 1.64 | 16.94 |
| | 98 | 108 | 267 | 1.98 | 50.45 | 461 | 1.58 | 17.07 |

Table 2, below, shows the percentage mass loss of water from the samples conditioned at 98% relative humidity.

TABLE 2
Percentage mass loss due to absorbed water on the surface of the
cellulose nanoparticle and aerogel samples.

| | Initial mass | Mass at | |
|---|---|---|---|
| Sample | (mg) | 120° C. | Mass loss (%) |
| Cellulose nanoparticles | 14.73 | 11.63 | 21.07 |
| 80 mg aerogel | 5.17 | 4.13 | 20.10 |
| 90 mg aerogel | 9.31 | 7.17 | 23.07 |
| 100 mg aerogel | 15.72 | 11.40 | 27.52 |
| 120 mg aerogel | 10.09 | 8.04 | 20.39 |
| 140 mg aerogel | 11.34 | 8.94 | 21.18 |
| 160 mg aerogel | 10.73 | 7.53 | 29.84 |

1. A cellulose aerogel comprising:
a plurality of cellulose nanoparticles.

2. The cellulose nanoparticle aerogel according to claim 1, wherein the cellulose nanoparticles comprise at least 50% cellulose nanocrystals by weight of cellulose nanoparticles.

3. The cellulose nanoparticle aerogel according to claim 1, wherein the cellulose nanoparticles comprise at least 80% cellulose nanocrystals by weight of cellulose nanoparticles.

4. The cellulose nanoparticle aerogel according to claim 1, wherein the cellulose nanoparticle aerogel has a density of from 0.001 to 0.2 g/cm³.

5. The cellulose nanoparticle aerogel according to claim 1, wherein the cellulose nanoparticle aerogel has a density of from 0.2 to 1.59 g/cm³.

6. The cellulose nanoparticle aerogel according to claim 1, wherein the cellulose nanoparticle aerogel has an average pore diameter of less than 100 nm.

7. The cellulose nanoparticle aerogel according to claim 1, wherein the cellulose nanoparticles comprise anionic and/or cationic surface groups.

8. The cellulose nanoparticle aerogel according to claim 1, wherein the cellulose nanoparticle aerogel exhibits adsorption hysteresis.

9. The cellulose nanoparticle aerogel according to claim 1, further comprising one or more carbon nanotubes.

10. The cellulose nanoparticle aerogel according to claim 9, wherein the cellulose nanoparticle aerogel comprises at least 1 wt % carbon nanotubes.

11. The cellulose nanoparticle aerogel according to claim 1, further comprising one or more metal, metal complexes metal nanoparticles, metal containing nanoparticles, metal oxides or metal alloy nanoparticles.

12. The cellulose nanoparticle aerogel according to claim 11, wherein the metal nanoparticles are selected from the group consisting of gold, silver, palladium and platinum nanoparticles and PbS quantum dots.

13. A cellulose hydrogel comprising:
a plurality of cellulose nanoparticles and water.

14. A cellulose organogel comprising:
a plurality of cellulose nanoparticles and one or more organic solvents.

15. A method of manufacturing a cellulose nanoparticle aerogel comprising:
forming one of a cellulose hydrogel comprising a plurality of cellulose nanoparticles and a cellulose organogel comprising a plurality of cellulose nanoparticles,
drying the cellulose nanoparticle hydrogel or organogel to form a cellulose nanoparticle aerogel.

16. The method of claim 15 wherein the cellulose hydrogel is formed by mixing a plurality of cellulose nanoparticles in water.

17. The method of claim 15 wherein the cellulose organogel is formed by mixing a plurality of cellulose nanoparticles in one or more organic solvents or by exchanging the water in a cellulose hydrogel comprising a plurality of cellulose nanoparticles with one or more organic solvents.

18. The method of claim 16, wherein the mixing comprises sonication, stirring, high shear mixing, shaking and/or tumbling.

19. The method of claim 15, wherein the hydrogel or organogel is prepared in a concentration of cellulose nanoparticles in water, or one or more organic solvents respectively, of at least 4 wt %.

20. The method of claim 17, wherein the one or more organic solvents is one or more of ethanol, methanol, propan-1-ol, propan-2-ol, n-butanol, sec-butanol, iso-butanol, and tert-butanol, acetone, THF, cyclohexane, NMP, and DMF.

21. The method of claim 15, wherein where a hydrogel is formed the hydrogel is dried by first exchanging the water in the hydrogel for one or more organic solvents that is miscible with water to form an organogel.

22. The method of claim 15, wherein where an organogel is formed, the one or more organic solvents of the organogel is miscible with a supercritical fluid.

23. The method of claim 22, wherein the drying of the organogel to form a cellulose nanoparticle aerogel is performed by supercritical drying with a supercritical fluid.

24. The method of claim 23, wherein the supercritical fluid is one or more of supercritical carbon dioxide, supercritical ethanol, supercritical methanol, supercritical methane, supercritical ethane, and supercritical helium.

25. The method of claim 15, wherein drying comprises freeze-drying or subcritically-drying the cellulose nanoparticle hydrogel or organogel to form a cellulose nanoparticle aerogel.

26. The method of claim 15, further comprising:
forming the cellulose nanoparticle aerogel in a material for use as, a thermal, sound or electrical insulator, a catalyst, a catalyst support, an electronics component, a particle filter, a storage media for substances, a coating, a rheology modifier, a thickening agent, a controlled release agent, an IR suppressant, a surface functionality to bind metals to provide reoriginalable porous catalyst supports, a scaffold for cell growth, in drug delivery, medicinal, water treatment, air disinfection or purification, gas adsorption or separation, metal sequestration or cosmetic applications, or in space and particle research.

27. The method of claim 15, further comprising:
forming the cellulose nanoparticle aerogel in textiles and outdoor apparel, sports equipment, in the absorption of materials, for example oil, and for use in or as windows.

28. The method of claim 15, further comprising:
forming the cellulose nanoparticle aerogel in gas and electrochemical storage media, supercapacitors, molecular computing elements, molecular-filtration membranes, artificial muscles, sensors, heterogeneous catalyst supports or nanotube-blended plastics, the cellulose nanoparticle aerogel comprising carbon nanotubes.

29. The method of claim 15, further comprising:
forming the cellulose nanoparticle aerogel in electronic, magnetic, mechanical, catalytic or composite materials; sensors; ultrafast data communication; catalytic materials; optical data storage; photovoltaics; biological nanosensors; electrochemistry; magnetic, biomedical or optoelectronic nanodevices; antimicrobial or biological applications such as wound dressing materials, body wall repairs, augmentation devices, tissue scaffolds or antimicrobial filters; catalysts for fuel cells; pharmaceutical drug additives, pharmaceutical devices; the storage of substances; the capture and storage of substances; water treatment; or air purification or disinfection, insulation, wherein the cellulose nanoparticle aerogel comprises one or more metal, metal complexes metal nanoparticles, metal containing nanoparticles, metal oxides, or metal alloy nanoparticles.

30. The method of claim 15, further comprising:
forming a container enclosing the cellulose nanoparticle aerogel.

31. The method of claim 15, further comprising:
forming a reoriginalable porous catalyst support comprising the cellulose nanoparticle aerogel with metallic groups, organic catalysts or enzymes immobilised on the aerogel.

32. The method of claim 15, further comprising:
forming a particle filter comprising the cellulose nanoparticle aerogel.

33. The method of claim 15, in which one of a thermal gravimetric analysis (TGA) and differential thermal gravimetric (DTG) analysis profiles of aerogel produced is substantially similar to that of the cellulose particles used to make the aerogel.

34. The method of claim 33, wherein the TGA and/or DTG analysis are performed at 06 and/or 98% relative humidity.
